SEQUENCE LISTING (15 sequences)

SEQ ID NO: 1 - DNA, 1149 bp, Artificial sequence
  synthesized sequence
atgagacgcacgaccttcaggaccatcgcctcgaccgccgcagcactgatgctgacgtcgggagcgctcaccgcgcacggggcgagcaccacgacggacgacaccgacggtgactacccagcggagatgctggctgccatggagtccgacctcgggctcagcgccgaccgggccaaggaacggctcgacttccaggtcgaggcgagcgagatccaggcgcagctcgacaaggagctgggcgaggacttcgccggcacctggctcgacgccgagagcgacgaggtcttcgtcgcgacgaccgaccgcggcaggtcgggcgcggtccgcgaccagggcgcacgggccgtcgtggtggagcactcgctcgagcagctgagcgagtggaccgacgagctgaccgaggcgctcgacggcgtcgacggcgtgccggggctgtacatcgacgtcgaggccaacggcctcgtcgtccaggtgcacgacggcgccacccgcgacgcacgccaggcggtgcgggacaccgacgtgccgcgccaggccgtcacctacgaggtgaccgacgaagagccggagctgtacaaccagggcggtgacgcctactacatcggcggtggcgggcgctgctcgatcgggttcgcggtgcagggcgggttcgtcaccgccggtcactgcgggtccaccggcgcctcgacgtcgtccccgaccggttcgttcgcggcgtcgcacttccccggcgacgacatggcgtgggtccgcaccagcgcgaccgtggccggcacggtcaacaactacagcggtggctcggtcgccgtccacggagcctccccagcgtccgtgggcgcccaggtctgccgttcaggctcgacgaccggctggcactgcgggaccatccaggcgttcaacgccacggtcacctacccgcagggcaccgtgcgcgggctcatccgcaccacggtgtgcgccgagcccggcgactccggcggctcgctcctggccggcaaccaggcccagggcgtgacctcgggcggctccggcaactgccgcaccggcgggaccaccttcttccagccggtccagccgatcctgcagcgctacggcttgtcgctcatcaccacgggcggcaacggtggcggcaacccgccgcccgcagcc

SEQ ID NO: 2 - protein, 383 aa, Artificial sequence
  Synthesized sequence
MRRTTFRTIASTAAALMLTSGALTAHGASTTTDDTDGDYPAEMLAAMESDLGLSADRAKERLDFQVEASEIQAQLDKELGEDFAGTWLDAESDEVFVATTDRGRSGAVRDQGARAVVVEHSLEQLSEWTDELTEALDGVDGVPGLYIDVEANGLVVQVHDGATRDARQAVRDTDVPRQAVTYEVTDEEPELYNQGGDAYYIGGGGRCSIGFAVQGGFVTAGHCGSTGASTSSPTGSFAASHFPGDDMAWVRTSATVAGTVNNYSGGSVAVHGASPASVGAQVCRSGSTTGWHCGTIQAFNATVTYPQGTVRGLIRTTVCAEPGDSGGSLLAGNQAQGVTSGGSGNCRTGGTTFFQPVQPILQRYGLSLITTGGNGGGNPPPAA

SEQ ID NO: 3 - DNA, 607 bp, Bacillus subtilis
aattctccattttcttctgctatcaaaataacagactcgtgattttccaaacgagctttcaaaaaagcctctgccccttgcaaatcggatgcctgtctataaaattcccgatattggttaaacagcggcgcaatggcggccgcatctgatgtctttgcttggcgaatgttcatcttatttcttcctccctctcaataattttttcattctatcccttttctgtaaagtttatttttcagaatacttttatcatcatgctttgaaaaaatatcacgataatatccattgttctcacggaagcacacgcaggtcatttgaacgaattttttcgacaggaatttgccgggactcaggagcatttaacctaaaaaagcatgacatttcagcataatgaacatttactcatgtctattttcgttcttttctgtatgaaaatagttatttcgagtctctacggaaatagcgagagatgatatacctaaatagagataaaatcatctcaaaaaaatgggtctactaaaatattattccatctattacaataaattcacagaatagtcttttaagtaagtctactctgaatttttttaaaaggagagggtaaaga

SEQ ID NO: 4 - protein, 29 aa, Bacillus subtilis
MRSKKLWISLLFALTLIFTMAFSNMSAQA

SEQ ID NO: 5 - DNA, 1167 bp, Artificial sequence
  Synthesized sequence
gtgagaagcaaaaaattgtggatcagcttgttgtttgcgttaacgttaatctttacgatggcgttcagcaacatgagcgcgcaggctgctggaaaagcatcaacaacaacggatgatacagatggcgattatccggcagaaatgctggcagcaatggaatcagatctgggcctgtcagcagatagagcaaaagaaagactggattttcaagttgaagcgagcgaaattcaagcgcaactggataaagaactgggcgaagattttgcaggcacatggctggatgcagaatcagatgaagtttttgttgcaacgacagatagaggcagatcaggcgcagttagagatcaaggcgcaagagcagttgttgtcgaacattcactggaacaactgtcagaatggacagatgaactgacggaagcactggatggcgttgatggcgtaccgggactgtatattgatgttgaagcaaatggcctggttgtccaagttcatgatggcgcaacaagagatgcaagacaagcagtcagagatacggatgttccgagacaagcggttacatatgaagttacagatgaagaaccggaactgtacaatcaaggcggagatgcgtattatatcggaggcggaggcagatgctcaattggctttgcagtccaaggcggatttgttacagcaggccattgcggctcaacaggcgcatcaacatcatcaccgacaggctcatttgcagcatcacattttccgggtgatgatatggcatgggttagaacatcagcaacagtcgcaggcacagttaataactattcaggcggatcagttgcagttcatggcgcatcaccggcatcagttggcgcacaagtttgcagaagcggcagcacaacaggctggcattgcggcacaattcaagcatttaatgcaacagttacatatccgcaaggcacagtcagaggcctgattagaacaacagtttgcgcagaacctggcgatagcggaggctcactgcttgcaggcaatcaagcacaaggcgttacatcaggcggaagcggcaattgcagaacaggcggaacaacattttttcaaccggttcaaccgattctgcaaagatatggcctgtcactgattacaacaggcggtaatggcggaggaaatcctcctccggcagcataa

SEQ ID NO: 6 - protein, 388 aa, Artificial sequence
  Synthesized sequence
MRSKKLWISLLFALTLIFTMAFSNMSAQAAGKASTTTDDTDGDYPAEMLAAMESDLGLSADRAKERLDFQVEASEIQAQLDKELGEDFAGTWLDAESDEVFVATTDRGRSGAVRDQGARAVVVEHSLEQLSEWTDELTEALDGVDGVPGLYIDVEANGLVVQVHDGATRDARQAVRDTDVPRQAVTYEVTDEEPELYNQGGDAYYIGGGGRCSIGFAVQGGFVTAGHCGSTGASTSSPTGSFAASHFPGDDMAWVRTSATVAGTVNNYSGGSVAVHGASPASVGAQVCRSGSTTGWHCGTIQAFNATVTYPQGTVRGLIRTTVCAEPGDSGGSLLAGNQAQGVTSGGSGNCRTGGTTFFQPVQPILQRYGLSLITTGGNGGGNPPPAA

SEQ ID NO: 7 - protein, 191 aa, Artificial sequence
  Synthesized sequence
NQGGDAYYIGGGGRCSIGFAVQGGFVTAGHCGSTGASTSSPTGSFAASHFPGDDMAWVRTSATVAGTVNNYSGGSVAVHGASPASVGAQVCRSGSTTGWHCGTIQAFNATVTYPQGTVRGLIRTTVCAEPGDSGGSLLAGNQAQGVTSGGSGNCRTGGTTFFQPVQPILQRYGLSLITTGGNGGGNPPPAA

SEQ ID NO: 8 - protein, 182 aa, Artificial sequence
  Synthesized sequence
NQGGDAYYIGGGGRCSIGFAVQGGFVTAGHCGSTGASTSSPTGSFAASHFPGDDMAWVRTSATVAGTVNNYSGGSVAVHGASPASVGAQVCRSGSTTGWHCGTIQAFNATVTYPQGTVRGLIRTTVCAEPGDSGGSLLAGNQAQGVTSGGSGNCRTGGTTFFQPVQPILQRYGLSLITTGGN

SEQ ID NO: 9 - protein, 27 aa, Artificial sequence
  Synthesized sequence
MRRTTFRTIASTAAALMLTSGALTAHG

SEQ ID NO: 10 - DNA, 247 bp, Bacillus amyloliquefaciens
acataaaaaaccggccttggccccgccggttttttattatttttcttcctccgcatgttcaatccgctccataatcgacggatggctccctctgaaaattttaacgagaaacggcgggttgacccggctcagtcccgtaacggccaagtcctgaaacgtctcaatcgccgcttcccggtttccggtcagctcaatgccgtaacggtcggcggcgttttcctgataccgggagacggcattcgtaatc

SEQ ID NO: 11 - DNA, 87 bp, Artificial sequence
  Synthesized sequence
atgatgaggaaaaagagtttttggcttgggatgctgacggccttcatgctcgtgttcacgatggcattcagcgattccgcttctgct

SEQ ID NO: 12 - DNA, 34 bp, Artificial sequence
  Synthesized sequence
cggatttcctgaaggaaatccgtttttttatttt

SEQ ID NO: 13 - protein, 187 aa, Artificial sequence
  Synthesized sequence
NQGGDAYYIGGGGRCSIGFAVQGGFVTAGHCGSTGASTSSPTGSFAASHFPGDDMAWVRTSATVAGTVNNYSGGSVAVHGASPASVGAQVCRSGSTTGWHCGTIQAFNATVTYPQGTVRGLIRTTVCAEPGDSGGSLLAGNQAQGVTSGGSGNCRTGGTTFFQPVQPILQRYGLSLITTGGNGGGNP

SEQ ID NO: 14 - protein, 269 aa, Bacillus subtilis
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 15 - protein, 275 aa, Bacillus amyloliquefaciens [sequence omitted]

(19) United States
(12) Patent Application Publication
(10) Pub. No.: US 2024/0174953 A1
(43) Pub. Date: May 30, 2024

(54) TRYPSIN-LIKE SERINE PROTEASES AND USES THEREOF

(71) Applicant: Danisco US Inc., Palo Alto, CA (US)

(72) Inventors: Lilia M Babe, Emerald Hills, CA (US); Xiaogang Gu, Shanghai (CN); Thijs Kaper, Half Moon Bay, CA (US)

(21) Appl. No.: 18/285,392

(22) PCT Filed: Apr. 21, 2022
(86) PCT No.: PCT/US2022/025707

§ 371 Date: Oct. 3, 2023

(30) Foreign Application Priority Data
Apr. 23, 2021 (WO) PCT/CN21/89333

(51) Int. Cl. C11D 3/386; C11D 1/72; C12N 9/50

(52) U.S. Cl. CPC C11D 3/38618; C11D 1/72; C12N 9/50; C12Y 304/21; C11D 2111/12; C11D 2111/14

(57) ABSTRACT

Described herein is at least one novel trypsin-like serine protease polypeptide and uses thereof. Further described herein are cleaning compositions containing at least one polypeptide described herein, wherein said composition can be used to clean fabrics and hard surfaces. Even further described herein is at least one cleaning composition selected from a laundry detergent, a dishwashing detergent (e.g., automatic and hand dish), and a personal care composition. Even still further, at least one polypeptide having improved soil removal and/or stability compared to at least one reference polypeptide is described herein.

This application is a 371 of International Application No. PCT/US2022/025707, filed Apr. 21, 2022 and claims the benefit of PCT/CN2021/089333, filed Apr. 23, 2021, the entire contents which are herein incorporated by reference in their entirety.

### REFERENCE TO SEQUENCE LISTING SUBMITTED ELECTRONICALLY

The official copy of the sequence listing is submitted electronically via EFS-Web as an ASCII formatted sequence listing with a file named 20230919_NB41883USPCT2_ST25 created on Sep. 19, 2023 and having a size of 22,254 bytes and is filed concurrently with the specification. The sequence listing contained in this ASCII formatted document is part of the specification and is herein incorporated by reference in its entirety.

### FIELD

Described herein is at least one novel trypsin-like serine protease polypeptide and uses thereof. Further described herein are cleaning compositions containing at least one polypeptide having serine protease activity described herein, wherein said composition can be used to clean fabrics or hard surfaces. Even further described herein is at least one cleaning composition selected from a laundry detergent, a dishwashing detergent (e.g., automatic and hand dish), and a personal care composition. Even still further, at least one polypeptide having serine protease activity and improved soil removal and/or stability compared to at least one reference polypeptide having serine protease activity is described herein.

### BACKGROUND

Proteases (also called peptidases or proteinases) are enzymes capable of cleaving peptide bonds. Proteases have evolved multiple times, and different classes of proteases can perform the same reaction by completely different catalytic mechanisms. Proteases can be found in animals, plants, fungi, bacteria, archaea and viruses.

Proteolysis can be achieved by enzymes currently classified into six broad groups: aspartyl proteases, cysteine proteases, serine proteases (such as, e.g., subtilisins or trypsin-like proteases), threonine proteases, glutamic proteases, and metalloproteases.

Serine proteases are a subgroup of carbonyl hydrolases comprising a diverse class of enzymes having a wide range of specificities and biological functions. Notwithstanding this functional diversity, the catalytic machinery of serine proteases has been approached by at least two genetically distinct families of enzymes: 1) the subtilisins; and 2) trypsin-like serine proteases (also known as chymotrypsin-related). These two families of serine proteases or serine endopeptidases have very similar catalytic mechanisms. The tertiary structure of these two enzyme families brings together a conserved catalytic triad of amino acids consisting of serine, histidine and aspartate.

Much research has been conducted on the serine proteases, in particular, subtilisins, due largely to their useful industrial applications. Additional work has been focused on adverse environmental conditions (e.g., exposure to oxidative agents, chelating agents, extremes of temperature and/or pH) which can adversely impact the functionality of these enzymes in a variety of applications.

Thus, there is a continuing need to find new serine proteases such as trypsin-like proteases of prokaryotic origins which can be used under adverse conditions and retain or have improved proteolytic activity and/or stability.

### SUMMARY

In a first embodiment, there is described at least one isolated polypeptide having serine protease activity, where the polypeptide comprises an amino acid sequence having at least 75%, 80%, 85%, 90%, 91%, 92%, 93%, 94%, 95%, 96%, 97%, 98%, 99%, or 100% sequence identity with an amino acid sequence selected from the group consisting of SEQ ID NO: 7, 8, and 13.

In another embodiment, the disclosure provides a recombinant construct comprising a regulatory sequence functional in a production host operably linked to a nucleotide sequence encoding at least one polypeptide having an amino acid sequence having at least 75%, 80%, 85%, 90%, 91%, 92%, 93%, 94%, 95%, 96%, 97%, 98%, 99%, or 100% sequence identity with an amino acid sequence selected from the group consisting of SEQ ID NOs: 7, 8, and 13.

In yet another embodiment, the disclosure provides host cells comprising a recombinant nucleic acid construct comprising an isolated polynucleotide comprising a nucleotide sequence that encodes a polypeptide having serine protease activity, wherein the polypeptide has at least 75%, 80%, 85%, 90%, 91%, 92%, 93%, 94%, 95%, 96%, 97%, 98%, 99%, or 100% sequence identity with an amino acid sequence selected from the group consisting of SEQ ID NOs: 7, 8, and 13 operably linked to a promoter sequence capable of controlling expression of the polynucleotide sequence.

A further embodiment provided herein includes, methods for producing at least one polypeptide comprising: (a) transforming a production host with the recombinant construct comprising a regulatory sequence functional in a production host operably linked to a nucleotide sequence encoding at least one polypeptide having an amino acid sequence having at least 75%, 80%, 85%, 90%, 91%, 92%, 93%, 94%, 95%, 96%, 97%, 98%, 99%, or 100% sequence identity with an amino acid sequence selected from the group consisting of SEQ ID NOs: 7, 8, and 13; and (b) culturing the production host of step (a) under conditions whereby at least one polypeptideis produced.

Also provided are compositions, including cleaning or detergent compositions, comprising at least one polypeptide having an amino acid sequence having at least 75%, 80%, 85%, 90%, 91%, 92%, 93%, 94%, 95%, 96%, 97%, 98%, 99%, or 100% sequence identity with an amino acid sequence selected from the group consisting of SEQ ID NOs: 7, 8, and 13 and, optionally, at least one surfactant and/or at least one dispersant.

In another embodiment, the disclosure provides methods of cleaning, comprising contacting a surface or an item in need of cleaning with an effective amount of at least one polypeptide having serine protease activity, where the polypeptide comprises an amino acid sequence having at least 75%, 80%, 85%, 90%, 91%, 92%, 93%, 94%, 95%, 96%, 97%, 98%, 99%, or 100% sequence identity with an amino acid sequence selected from the group consisting of SEQ ID NO: 7, 8, and 13, or a composition comprising such a polypeptide; and optionally further comprising the step of rinsing said surface or item after contacting said surface or item with said polypeptide.

### BRIEF DESCRIPTION OF THE DRAWINGS AND SEQUENCES

FIG. 1. FIG. 1A provides a mass spectrogram of purified IspPro1. FIG. 1B Isolated fractions of one embodiment of purified IspPro1 sample separated by gel filtration. FIG. 1C Specific activity of isolated IspPro1 fractions shown in FIG. 1B on azo-casein.

FIG. 2. Provides a graphic representation of a dose response curves of IspPro1 in the pNA-AAPF assay.

FIG. 3. Provides a graphic representation of pH profile of purified IspPro1.

FIG. 4. Provides a graphic representation of the temperature profile of purified IspPro1.

FIG. 5. Provides a graphic representation of an example of cleaning performance of IspPro1 on PA-S-38 in GSM-D detergent at pH 10.9.

FIG. 6. FIG. 6A: Provides a graphic representation of an example of cleaning performance of IspPro1 on EMPA-116 in Tide HDL at pH 8.0/16° C. FIG. 6B: Provides an example of cleaning performance of IspPro1 on EMPA-116 in Tide HDL at pH 8.0/32° C.

FIG. 7. Provides a graphic representation of an example of cleaning performance of IspPro1 on C-S-39 in PNB HDL at pH 8.2

FIG. 8. FIG. 8A: Provides a graphic representation of an example of cleaning performance of IspPro1 on EMPA-116 in Tide HDD at pH 10.0/16° C. FIG. 8B: Provides a graphic representation of an example of cleaning performance of IspPro1 on EMPA-116 in Tide HDD at pH 10.0/32° C.

FIG. 9. Provides a graphic representation of an example of cleaning performance of IspPro1 on C-S-01 in Tide HDD.

FIG. 10. Provides a graphic representation of the results of an extended stability of IspPro1 in buffer.

FIG. 11. Provides a graphic representation of the results of a long term storage stability assay of IspPro1 in PNB detergent.

### DETAILED DESCRIPTION

In this disclosure, a number of terms and abbreviations are used. The following definitions apply unless specifically stated otherwise.

The articles “a”, “an”, and “the” preceding an element or component are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

The term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

As used herein in connection with a numerical value, the term “about” refers to a range of +/−0.5 of the numerical value, unless the term is otherwise specifically defined in context. For instance, the phrase a “pH value of about 6” refers to pH values of from 5.5 to 6.5, unless the pH value is specifically defined otherwise.

It is intended that every maximum numerical limitation given throughout this Specification includes every lower numerical limitation, as if such lower numerical limitations were expressly written herein. Every minimum numerical limitation given throughout this Specification will include every higher numerical limitation, as if such higher numerical limitations were expressly written herein. Every numerical range given throughout this Specification will include every narrower numerical range that falls within such broader numerical range, as if such narrower numerical ranges were all expressly written herein.

The term “protease” means a protein or polypeptide domain derived from a microorganism, e.g., a fungus, bacterium, or from a plant or animal, and that has the ability to catalyze cleavage of peptide bonds at one or more of various positions of a protein backbone (e.g., E.C. 3.4). The terms “protease”, “peptidase” and “proteinase” can be used interchangeably. Proteases can be found in animals, plants, fungi, bacteria, archaea and viruses. Proteolysis can be achieved by enzymes currently classified into six broad groups based on their catalytic mechanisms: aspartyl proteases, cysteine proteases, trypsin-like serine proteases, threonine proteases, glutamic proteases, and metalloproteases.

The term “serine protease” refers to enzymes that cleave peptide bonds in proteins, in which serine serves as the nucleophilic amino acid at the active site of the enzyme. Serine proteases fall into two broad categories based on their structure: the chymotrypsin-like (trypsin-like) and the subtilisins. In the MEROPS protease classification system, proteases are distributed among 16 superfamilies and numerous families. The family S8 includes the subtilisins and the family S1 includes the chymotrypsin-like (trypsin-like) enzymes. The subfamily SIE includes the trypsin-like serine proteases from Strepmocyces organisms, such as Streptogricins A, B and C. The terms “serine protease”, “trypsin-like serine protease” and “chymotrypsin-like protease” are used interchangeably herein.

The term “isolated” means a substance in a form or environment that does not occur in nature. Non-limiting examples of isolated substances include (1) any non- naturally occurring substance, (2) any substance including, but not limited to, any host cell, enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated. The terms “isolated nucleic acid molecule”, “isolated polynucleotide”, and “isolated nucleic acid fragment” will be used interchangeably and refer to a polymer of RNA or DNA that is single- or double-stranded, optionally containing synthetic, non-natural or altered nucleotide bases. An isolated nucleic acid molecule in the form of a polymer of DNA may be comprised of one or more segments of cDNA, genomic DNA or synthetic DNA.

The term “purified” as applied to nucleic acids or polypeptides generally denotes a nucleic acid or polypeptide that is essentially free from other components as determined by analytical techniques well known in the art (e.g., a purified polypeptide or polynucleotide forms a discrete band in an electrophoretic gel, chromatographic eluate, and/or a media subjected to density gradient centrifugation). For example, a nucleic acid or polypeptide that gives rise to essentially one band in an electrophoretic gel is “purified.” A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

As used herein, the term “functional assay” refers to an assay that provides an indication of a protein's activity. In some embodiments, the term refers to assay systems in which a protein is analyzed for its ability to function in its usual capacity. For example, in the case of a protease, a functional assay involves determining the effectiveness of the protease to hydrolyze a proteinaceous substrate.

The terms “peptides”, “proteins” and “polypeptides are used interchangeably herein and refer to a polymer of amino acids joined together by peptide bonds. A “protein” or “polypeptide” comprises a polymeric sequence of amino acid residues. The single and 3-letter code for amino acids as defined in conformity with the IUPAC-IUB Joint Commission on Biochemical Nomenclature (JCBN) is used throughout this disclosure. The single letter X refers to any of the twenty amino acids. It is also understood that a polypeptide may be coded for by more than one nucleotide sequence due to the degeneracy of the genetic code. Mutations can be named by the one letter code for the parent amino acid, followed by a position number and then the one letter code for the variant amino acid. For example, mutating glycine (G) at position 87 to serine (S) is represented as “G087S” or “G87S”. When describing modifications, a position followed by amino acids listed in parentheses indicates a list of substitutions at that position by any of the listed amino acids. For example, 6(L,I) means position 6 can be substituted with a leucine or isoleucine. At times, in a sequence, a slash (/) is used to define substitutions, e.g. F/V, indicates that the particular position may have a phenylalanine or valine at that position.

A “prosequence” or “propeptide sequence” refers to an amino acid sequence between the signal peptide sequence and mature protease sequence that is necessary for the proper folding and secretion of the protease; they are sometimes referred to as intramolecular chaperones. Prosequences may also follow the c-terminus of the mature sequence. Cleavage of the prosequence or propeptide sequence results in a mature active protease. Proteases are often expressed as pro-enzymes.

The terms “signal sequence” and “signal peptide” refer to a sequence of amino acid residues that may participate in the secretion or direct transport of the mature or precursor form of a protein. The signal sequence is typically located N-terminal to the precursor or mature protein sequence. The signal sequence may be endogenous or exogenous. A signal sequence is normally absent from the mature protein. A signal sequence is typically cleaved from the protein by a signal peptidase after the protein is transported.

The term “mature” form of a protein, polypeptide, or peptide refers to the functional form of the protein, polypeptide, or enzyme without the signal peptide sequence and propeptide sequence.

The term “precursor” form of a protein or peptide refers to a mature form of the protein having a prosequence operably linked to the amino or carbonyl terminus of the protein. The precursor may also have a “signal” sequence operably linked to the amino terminus of the prosequence. The precursor may also have additional polypeptides that are involved in post-translational activity (e.g., polypeptides cleaved therefrom to leave the mature form of a protein or peptide).

The term “wild-type” in reference to an amino acid sequence or nucleic acid sequence indicates that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

As used herein with regard to amino acid residue positions, “corresponding to” or “corresponds to” or “corresponds” refers to an amino acid residue at the enumerated position in a protein or peptide, or an amino acid residue that is analogous, homologous, or equivalent to an enumerated residue in a protein or peptide. As used herein, “corresponding region” generally refers to an analogous position in a related proteins or a reference protein.

The terms “derived from” and “obtained from” refer to not only a protein produced or producible by a strain of the organism in question, but also a protein encoded by a DNA sequence isolated from such strain and produced in a host organism containing such DNA sequence. Additionally, the term refers to a protein which is encoded by a DNA sequence of synthetic and/or cDNA origin and which has the identifying characteristics of the protein in question.

The term “reference”, with respect to a polypeptide described herein, refers to a naturally-occurring polypeptide that does not include a man-made substitution, insertion, or deletion at one or more amino acid positions, as well as a naturally-occurring or synthetic polypeptide that includes one or more man-made substitutions, insertions, or deletions at one or more amino acid positions. Similarly, the term “reference”, with respect to a polynucleotide, refers to a naturally-occurring polynucleotide that does not include a man-made substitution, insertion, or deletion of one or more nucleosides, as well as a naturally-occurring or synthetic polynucleotide that includes one or more man-made substitutions, insertions, or deletions at one or more nucleosides. For example, a polynucleotide encoding a wild-type or parental polypeptide is not limited to a naturally-occurring polynucleotide, and encompasses any polynucleotide encoding the wild-type or parental polypeptide.

It would be recognized by one of ordinary skill in the art that modifications of amino acid sequences disclosed herein can be made while retaining the function associated with the disclosed amino acid sequences. For example, it is well known in the art that alterations in a gene which result in the production of a chemically equivalent amino acid at a given site, but do not affect the functional properties of the encoded protein are common. For example, any particular amino acid in an amino acid sequence disclosed herein may be substituted for another functionally equivalent amino acid. For the purposes of this disclosure, substitutions are defined as exchanges within one of the following five groups:
1. Small aliphatic, nonpolar or slightly polar residues: Ala, Ser, Thr (Pro, Gly);
2. Polar, negatively charged residues and their amides: Asp, Asn, Glu, Gln;
3. Polar, positively charged residues: His, Arg, Lys;
4. Large aliphatic, nonpolar residues: Met, Leu, Ile, Val (Cys); and
5. Large aromatic residues: Phe, Tyr, and Trp.

Thus, a codon for the amino acid alanine, a hydrophobic amino acid, may be substituted by a codon encoding another less hydrophobic residue (such as glycine) or a more hydrophobic residue (such as valine, leucine, or isoleucine). Similarly, changes which result in substitution of one negatively charged residue for another (such as serine acid for glutamic acid) or one positively charged residue for another (such as lysine for arginine) can also be expected to produce a functionally equivalent product. In many cases, nucleotide changes which result in alteration of the N-terminal and C-terminal portions of the protein molecule would also not be expected to alter the activity of the protein. Each of the proposed modifications is well within the routine skill in the art, as is determination of retention of biological activity of the encoded products.

The term “codon optimized”, as it refers to genes or coding regions of nucleic acid molecules for transformation of various hosts, refers to the alteration of codons in the gene or coding regions of the nucleic acid molecules to reflect the typical codon usage of the host organism without altering the polypeptide for which the DNA codes.

The term “gene” refers to a nucleic acid molecule that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different from that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

The term “coding sequence” refers to a nucleotide sequence which codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding sites, and stem-loop structures.

The term “operably linked” refers to the association of nucleic acid sequences on a single nucleic acid molecule so that the function of one is affected by the other. For example, a promoter is operably linked with a coding sequence when it is capable of affecting the expression of that coding sequence, i.e., the coding sequence is under the transcriptional control of the promoter. Coding sequences can be operably linked to regulatory sequences in sense or antisense orientation.

The terms “regulatory sequence” or “control sequence” are used interchangeably herein and refer to a segment of a nucleotide sequence which is capable of increasing or decreasing expression of specific genes within an organism. Examples of regulatory sequences include, but are not limited to, promoters, signal sequence, operators and the like. As noted above, regulatory sequences can be operably linked in sense or antisense orientation to the coding sequence/gene of interest.

“Promoter” or “promoter sequences” refer to DNA sequences that define where transcription of a gene by RNA polymerase begins. Promoter sequences are typically located directly upstream or at the 5′ end of the transcription initiation site. Promoters may be derived in their entirety from a native or naturally occurring sequence, or be composed of different elements derived from different promoters found in nature, or even comprise synthetic DNA segments. It is understood by those skilled in the art that different promoters may direct the expression of a gene in different tissues or cell type or at different stages of development, or in response to different environmental or physiological conditions (“inducible promoters”). The “3′ non-coding sequences” refer to DNA sequences located downstream of a coding sequence and include sequences encoding regulatory signals capable of affecting mRNA processing or gene expression, such as termination of transcription.

The term “transformation” as used herein refers to the transfer or introduction of a nucleic acid molecule into a host organism. The nucleic acid molecule may be introduced as a linear or circular form of DNA. The nucleic acid molecule may be a plasmid that replicates autonomously, or it may integrate into the genome of a production host. Production hosts containing the transformed nucleic acid are referred to as “transformed” or “recombinant” or “transgenic” organisms or “transformants”.

The term “recombinant” as used herein refers to an artificial combination of two otherwise separated segments of nucleic acid sequences, e.g., by chemical synthesis or by the manipulation of isolated segments of nucleic acids by genetic engineering techniques. For example, DNA in which one or more segments or genes have been inserted, either naturally or by laboratory manipulation, from a different molecule, from another part of the same molecule, or an artificial sequence, resulting in the introduction of a new sequence in a gene and subsequently in an organism. The terms “recombinant”, “transgenic”, “transformed”, “engineered” or “modified for exogenous gene expression” are used interchangeably herein.

The terms “recombinant construct”, “expression construct”, “ recombinant expression construct” and “expression cassette” are used interchangeably herein. A recombinant construct comprises an artificial combination of nucleic acid fragments, e.g., regulatory and coding sequences that are not all found together in nature. For example, a construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:2411-2418; De Almeida et al., (1989) Mol Gen Genetics 218:78-86), and thus that multiple events are typically screened in order to obtain lines displaying the desired expression level and pattern. Such screening may be accomplished standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The terms “production host”, “host” and “host cell” are used interchangeably herein and refer to any organism, or cell thereof, whether human or non-human into which a recombinant construct can be stably or transiently introduced in order to express a gene. This term encompasses any progeny of a parent cell, which is not identical to the parent cell due to mutations that occur during propagation.

The term “percent identity” is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the number of matching nucleotides or amino acids between strings of such sequences. “Identity” and “similarity” can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Methods to determine identity and similarity are codified in publicly available computer programs.

As used herein, “% identity” or percent identity” or “PID” refers to protein sequence identity. Percent identity may be determined using standard techniques known in the art. Useful algorithms include the BLAST algorithms (See, Altschul et al., J Mol Biol, 215:403-410, 1990; and Karlin and Altschul, Proc Natl Acad Sci USA, 90:5873-5787, 1993). The BLAST program uses several search parameters, most of which are set to the default values. The NCBI BLAST algorithm finds the most relevant sequences in terms of biological similarity but is not recommended for query sequences of less than 20 residues (Altschul et al., Nucleic Acids Res, 25:3389-3402, 1997; and Schaffer et al., Nucleic Acids Res, 29:2994-3005, 2001). Exemplary default BLAST parameters for a nucleic acid sequence searches include: Neighboring words threshold=11; E-value cutoff=10; Scoring Matrix =NUC.3.1 (match=1, mismatch =- 3);Gap Opening=5; and Gap Extension=2. Exemplary default BLAST parameters for amino acid sequence searches include: Word size=3; E-value cutoff=10; Scoring Matrix=BLOSUM62; Gap Opening=11; and Gap extension=1. A percent (%) amino acid sequence identity value is determined by the number of matching identical residues divided by the total number of residues of the “reference” sequence including any gaps created by the program for optimal/maximum alignment. BLAST algorithms refer to the “reference” sequence as the “query” sequence.

As used herein, “homologous proteins” or “homologous proteases” refers to proteins that have distinct similarity in primary, secondary, and/or tertiary structure. Protein homology can refer to the similarity in linear amino acid sequence when proteins are aligned. Homologous search of protein sequences can be done using BLASTP and PSI-BLAST from NCBI BLAST with threshold (E-value cut-off) at 0.001. (Altschul SF, Madde TL, Shaffer AA, Zhang J, Zhang Z, Miller W, Lipman DJ. Gapped BLAST and PSI BLAST a new generation of protein database search programs. Nucleic Acids Res 1997 Set 1;25(17):3389-402). Using this information, proteins sequences can be grouped. A phylogenetic tree can be built using the amino acid sequences.

The phrase “substantially-free of boron” refers to a composition or formulation that contains trace amounts of boron, for example, less than about 1000 ppm (1 mg/kg or liter equals 1 ppm), less than about 100 ppm, less than about 50 ppm, less than about 10 ppm, or less than about 5 ppm, or less than about 1 ppm. The trace amounts of boron may be present in the composition or formulation through, for example, the addition of other components containing trace amounts of boron and not by virtue of intentional addition to the detergent or formulation.

The term “cleaning activity” refers to cleaning performance achieved by a reference protease or one or more polypeptide described herein under conditions prevailing during the proteolytic, hydrolyzing, cleaning, or other process described herein. In some embodiments, cleaning performance of a reference protease or one or more polypeptide described herein may be determined by using one or more assay directed to cleaning one or more enzyme sensitive stain on an item or surface (e.g., a stain resulting from food, grass, blood, ink, milk, oil, and/or egg protein). Cleaning performance of a reference protease or one or more polypeptide described herein can be determined by subjecting the stain on an item or surface to standard wash condition(s) and assessing the degree to which the stain is removed by using various chromatographic, spectrophotometric, or other quantitative methodologies. Exemplary cleaning assays and methods are known in the art and include, but are not limited to those described in WO99/34011 and U.S. Pat. No. 6,605,458, as well as those cleaning assays and methods included in the Examples provided below.

The term “cleaning effective amount” of a reference protease or one or more polypeptide described herein refers to the amount of protease or one or more polypeptide described herein that achieves the desired level of enzymatic activity in the cleaning composition. Such effective amounts are readily ascertained by one of ordinary skill in the art and are based on many factors, such as the particular protease used, the cleaning application, the formulation of the cleaning composition, and whether a liquid or dry (e.g., granular, tablet, bar) composition is required, etc.

The term “cleaning adjunct material” refers to any liquid, solid, or gaseous material included in cleaning composition other than the one or more polypeptide described herein. In some embodiments, one or more cleaning composition described herein includes one or more cleaning adjunct material. Each cleaning adjunct material is typically selected depending on the particular type and form of cleaning composition (e.g., liquid, granule, powder, bar, paste, spray, tablet, gel, foam, or other composition). Preferably, each cleaning adjunct material is compatible with the one or more polypeptide described herein.

Sequence alignments and percent identity calculations may be performed using the Megalign program of the LASERGENE bioinformatics computing suite (DNASTAR Inc., Madison, WI), the AlignX program of Vector NTI v. 7.0 (Informax, Inc., Bethesda, MD), or the EMBOSS Open Software Suite (EMBL-EBI; Rice et al., Trends in Genetics 16, (6):276-277 (2000)). Multiple alignment of the sequences can be performed using the CLUSTAL method (such as CLUSTALW; for example version 1.83) of alignment (Higgins and Sharp, CABIOS, 5:151-153 (1989); Higgins et al., Nucleic Acids Res. 22:4673-4680 (1994); and Chenna et al., Nucleic Acids Res 31 (13):3497-500 (2003)), available from the European Molecular Biology Laboratory via the European Bioinformatics Institute) with the default parameters. Suitable parameters for CLUSTALW protein alignments include GAP Existence penalty=15, GAP extension −0.2, matrix=Gonnet (e.g., Gonnet250), protein ENDGAP=−1, protein GAPDIST=4, and KTUPLE=1. In one embodiment, a fast or slow alignment is used with the default settings where a slow alignment. Alternatively, the parameters using the CLUSTALW method (e.g., version 1.83) may be modified to also use KTUPLE=1, GAP PENALTY=10, GAP extension=1, matrix=BLOSUM (e.g., BLOSUM64), WINDOW=5, and TOP DIAGONALS SAVED=5. The MUSCLE program (Robert C. Edgar. MUSCLE: multiple sequence alignment with high accuracy and high throughput Nucl. Acids Res. (2004) 32 (5): 1792-1797) is yet another example of a multiple sequence alignment algorithm.

Various polypeptide amino acid sequences and polynucleotide sequences are disclosed herein. Variants of these sequences that are at least about 70-85%, 85-90%, or 90%-95% identical to the sequences disclosed herein may be used in certain embodiments. Alternatively, a variant polypeptide sequence or polynucleotide sequence in certain embodiments can have at least 60%, 61%, 62%,63%,64%, 65%, 66%, 67%, 68%,69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity with a sequence disclosed herein. The variant amino acid sequence or polynucleotide sequence has the same function of the disclosed sequence, or at least about 85%, 86%, 87%, 88%, 89%, 90%, 91%, 92%, 93%, 94%, 95%, 96%, 97%, 98%, or 99% of the function of the disclosed sequence.

The term “variant”, with respect to a polypeptide, refers to a polypeptide that differs from a specified wild-type, parental, or reference polypeptide in that it includes one or more naturally-occurring or man-made substitutions, insertions, or deletions of an amino acid. Similarly, the term “variant,” with respect to a polynucleotide, refers to a polynucleotide that differs in nucleotide sequence from a specified wild-type, parental, or reference polynucleotide. The identity of the wild-type, parental, or reference polypeptide or polynucleotide will be apparent from context.

The terms “plasmid”, “vector” and “cassette” refer to an extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector containing a gene and having elements in addition to the gene that facilitates transformation of a particular host cell. The terms “expression cassette” and “expression vector are used interchangeably herein and refer to a specific vector containing a gene and having elements in addition to the gene that allow for expression of that gene in a host.

The term “expression”, as used herein, refers to the production of a functional end-product (e.g., an mRNA or a protein) in either precursor or mature form. Expression may also refer to translation of mRNA into a polypeptide.

Expression of a gene involves transcription of the gene and translation of the mRNA into a precursor or mature protein. “Mature” protein refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be but are not limited to intracellular localization signals. “Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance. Host organisms containing the transformed nucleic acid fragments are referred to as “transgenic” organisms

The expression vector can be one of any number of vectors or cassettes useful for the transformation of suitable production hosts known in the art. Typically, the vector or cassette will include sequences directing transcription and translation of the relevant gene, a selectable marker, and sequences allowing autonomous replication or chromosomal integration. Suitable vectors generally include a region 5′ of the gene which harbors transcriptional initiation controls and a region 3′ of the DNA fragment which controls transcriptional termination. Both control regions can be derived from homologous genes to genes of a transformed production host cell and/or genes native to the production host, although such control regions need not be so derived.

Possible initiation control regions or promoters that can be included in the expression vector are numerous and familiar to those skilled in the art. Virtually any promoter capable of driving these genes is suitable, including but not limited to, CYC1, HIS3, GALI, GAL10, ADHI, PGK, PHO5, GAPDH, ADC1, TRP1, URA3, LEU2, ENO, TPI (useful for expression in Saccharomyces); AOXI (useful for expression in Pichia); and lac, araB, tet, trp, lP_{L}, lP_{R}, T7, tac, and trc (useful for expression in Escherichia coli) as well as the amy, apr, npr promoters and various phage promoters useful for expression in Bacillus. In some embodiments, the promoter is a constitutive or inducible promoter. A “constitutive promoter” is a promoter that is active under most environmental and developmental conditions. An “inducible” or “repressible” promoter is a promoter that is active under environmental or developmental regulation. In some embodiments, promoters are inducible or repressible due to changes in environmental factors including but not limited to, carbon, nitrogen or other nutrient availability, temperature, pH, osmolarity, the presence of heavy metal(s), the concentration of inhibitor(s), stress, or a combination of the foregoing, as is known in the art. In some embodiments, the inducible or repressible promoters are inducible or repressible by metabolic factors, such as the level of certain carbon sources, the level of certain energy sources, the level of certain catabolites, or a combination of the foregoing as is known in the art. In one embodiment, the promoter is one that is native to the host cell. For example, when T. reesei is the host, the promoter is a native T. reesei promoter such as the cbh1 promoter which is deposited in GenBank under Accession Number D86235.

Suitable non-limiting examples of promoters include cbh1, cbh2, egl1, egl2, egl3, egl4, egl5, xyn1, and xyn2, repressible acid phosphatase gene (phoA) promoter of P. chrysogenus (see e.g., Graessle et al., (1997) Appl. Environ. Microbiol., 63 :753-756), glucose repressible PCK1 promoter (see e.g., Leuker et al., (1997), Gene, 192:235-240), maltose inducible, glucose-repressible MET3 promoter (see Liu et al., (2006), Eukary. Cell, 5:638-649), pKi promoter and cpc1 promoter. Other examples of useful promoters include promoters from A. awamori and A. niger glucoamylase genes (see e.g., Nunberg et al., (1984) Mol. Cell Biol. 15 4:2306-2315 and Boel et al., (1984) EMBO J. 3:1581-1585). Also, the promoters of the T. reesei xln1 gene may be useful (see e.g., EPA 137280A1).

DNA fragments which control transcriptional termination may also be derived from various genes native to a preferred production host cell. In certain embodiments, the inclusion of a termination control region is optional. In certain embodiments, the expression vector includes a termination control region derived from the preferred host cell.

The expression vector can be included in the production host, particularly in the cells of microbial production hosts. The production host cells can be microbial hosts found within the fungal or bacterial families and which grow over a wide range of temperature, pH values, and solvent tolerances. For example, it is contemplated that any of bacteria, algae, and fungi such as filamentous fungi and yeast may suitably host the expression vector.

Inclusion of the expression vector in the production host cell may be used to express the protein of interest so that it may reside intracellularly, extracellularly, or a combination of both inside and outside the cell. Extracellular expression renders recovery of the desired protein from a fermentation product more facile than methods for recovery of protein produced by intracellular expression.

Certain embodiments relate to an isolated polypeptide having serine protease activity, selected from a polypeptide comprising an amino acid sequence with at least 60%, 65%, 70%, 75%, 80%, 85%, 90%, 91%, 92%, 93%, 94%, 95%, 96%, 97%, 98%, 99% or 100% amino acid sequence identity with SEQ ID NOs: 2, 6, 7, 8, and 13.

Other embodiments include a recombinant construct comprising a regulatory sequence functional in a production host operably linked to a nucleotide sequence encoding at least one polypeptide selected from: a polypeptide comprising an amino acid sequence with at least 60%, 65%, 70%, 75%, 80%, 85%, 90%, 91%, 92%, 93%, 94%, 95%, 96%, 97%, 98%, 99% or 100% amino acid sequence identity with SEQ ID NOs: 2, 6, 7, 8, and 13. In some embodiments, the production host is selected from the group consisting of fungi, bacteria, and algae. In other embodiments, the production host is used to produce at least one polypeptide described herein comprising: (a) transforming a production host with the recombinant construct described herein; and (b) culturing the production host of step (a) under conditions whereby at least one polypeptide described herein is produced. According to this method, at least one polypeptide described herein is optionally recovered from the production host. In another aspect, a serine protease-containing culture supernatant is obtained by using any of the methods described herein.

Also described herein is a recombinant microbial production host for expressing at least one polypeptide described herein, said recombinant microbial production host comprising a recombinant construct described herein. In another embodiment, this recombinant microbial production host is selected from the group consisting of bacteria, fungi and algae.

Expression will be understood to include any step involved in producing at least one polypeptide described herein including, but not limited to, transcription, post-transcriptional modification, translation, post-translation modification and secretion.

Techniques for modifying nucleic acid sequences utilizing cloning methods are well known in the art.

A polynucleotide encoding a trypsin-like serine protease can be manipulated in a variety of ways to provide for expression of the polynucleotide in a Bacillus host cell. Manipulation of the polynucleotide sequence prior to its insertion into a nucleic acid construct or vector may be desirable or necessary depending on the nucleic acid construct or vector or the Bacillus host cell. The techniques for modifying nucleotide sequences utilizing cloning methods are well known in the art.

Regulatory sequences are defined above. They include all components, which are necessary or advantageous for the expression of a trypsin-like serine protease. Each control sequence may be native or foreign to the nucleotide sequence encoding the trypsin-like serine protease. Such regulatory sequences include, but are not limited to, a leader, a polyadenylation sequence, a propeptide sequence, a promoter, a signal sequence and a transcription terminator. Regulatory sequences may be provided with linkers for the purpose of introducing specific restriction sites facilitating ligation or the regulatory sequences with the coding region of the nucleotide sequence encoding a trypsin-like serine protease.

A nucleic acid construct comprising a polynucleotide encoding a trypsin-like serine protease may be operably linked to one or more control sequences capable of directing the expression of the coding sequence in a Bacillus host cell under conditions compatible with the control sequences.

Each control sequence may be native or foreign to the polynucleotide encoding a trypsin-like serine protease. Such control sequences include, but are not limited to, a leader, a promoter, a signal sequence, and a transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a trypsin-like serine protease.

The control sequence may be an appropriate promoter region, a nucleotide sequence that is recognized by a Bacillus host cell for expression of the polynucleotide encoding a a trypsin-like serine protease. The promoter region contains transcription control sequences that mediate the expression of a trypsin-like serine protease. The promoter region may be any nucleotide sequence that shows transcriptional activity in the Bacillus host cell of choice and may be obtained from genes directing synthesis of extracellular or intracellular polypeptides having biological activity either homologous or heterologous to the Bacillus host cell.

The promoter region may comprise a single promoter or a combination of promoters. Where the promoter region comprises a combination of promoters, the promoters are preferably in tandem. A promoter of the promoter region can be any promoter that can initiate transcription of a polynucleotide encoding a polypeptide having biological activity in a Bacillus host cell of interest. The promoter may be native, foreign, or a combination thereof, to the nucleotide sequence encoding a polypeptide having biological activity. Such a promoter can be obtained from genes directing synthesis of extracellular or intracellular polypeptides having biological activity either homologous or heterologous to the Bacillus host cell.

Thus, in certain embodiments, the promoter region comprises a promoter obtained from a bacterial source. In other embodiments, the promoter region comprises a promoter obtained from a Gram positive or Gram negative bacterium. Gram positive bacteria include, but are not limited to, Bacillus, Streptococcus, Streptomyces, Staphylococcus, Enterococcus, Lactobacillus, Lactococcus, Clostridium, Geobacillus, and Oceanobacillus. Gram negative bacteria include, but are not limited to, E. coli, Pseudomonas, Salmonella, Campylobacter, Helicobacter, Flavobacterium, Fusobacterium, Ilyobacter, Neisseria, and Ureaplasma.

The promoter region may comprise a promoter obtained from a Bacillus strain (e.g., Bacillus agaradherens, Bacillus alkalophilus, Bacillus amyloliquefaciens, Bacillus brevis, Bacillus circulans, Bacillus clausii, Bacillus coagulans, Bacillus firmus, Bacillus gibsonii, Bacillus lautus, Bacillus lentus, Bacillus licheniformis, Bacillus megaterium, Bacillus pumilus, Bacillus stearothermophilus, Bacillus subtilis, or Bacillus thuringiensis; or from a Streptomyces strain (e.g., Streptomyces lividans or Streptomyces murinus).

Examples of suitable promoters for directing transcription of a polynucleotide encoding a polypeptide having biological activity in the methods of the present disclosure are the promoters obtained from the E. coli lac operon, Streptomyces coelicolor agarase gene (dagA), Bacillus lentus or Bacillus clausii alkaline protease gene (aprH), Bacillus licheniformis alkaline protease gene (subtilisin Carlsberg gene), Bacillus subtilis levansucrase gene (sacB), Bacillus subtilis alpha-amylase gene (amyE), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, Bacillus thuringiensis subsp. tenebfionis CryIIIA gene (cryIIIA) or portions thereof, prokaryotic beta-lactamase gene (Villa-Kamaroff et al., 1978, Proceedings of the National Academy of Sciences USA 75:3727-3731), and Bacillus megaterium xylA gene (Rygus and Hillen, 1992, J. Bacteriol. 174: 3049-3055; Kim et al., 1996, Gene 181: 71-76). Other examples are the promoter of the spol bacterial phage promoter and the tac promoter (DeBoer et al., 1983, Proceedings of the National Academy of Sciences USA 80:21-25). Further promoters are described in “Useful proteins from recombinant bacteria” in Scientific American, 1980, 242:74-94; and in Sambrook, Fritsch, and Maniatis, 1989, Molecular Cloning, A Laboratory Manual, 2d edition, Cold Spring Harbor, N.Y.

The promoter region may comprise a promoter that is a “consensus” promoter having the sequence TTGACA for the “-35” region and TATAAT for the “-10” region. The consensus promoter may be obtained from any promoter that can function in a Bacillus host cell. The construction of a “consensus” promoter may be accomplished by site-directed mutagenesis using methods well known in the art to create a promoter that conforms more perfectly to the established consensus sequences for the “-10” and “-35” regions of the vegetative “sigma A-type” promoters for Bacillus subtilis (Voskuil et al., 1995, Molecular Microbiology 17: 271-279).

A control sequence may also be a suitable transcription terminator sequence, such as a sequence recognized by a Bacillus host cell to terminate transcription. The terminator sequence is operably linked to the 3′ terminus of the nucleotide sequence encoding a trypsin-like serine protease. Any terminator that is functional in the Bacillus host cell may be used.

The control sequence may also be a suitable leader sequence, a non-translated region of a mRNA that is important for translation by a Bacillus host cell. The leader sequence is operably linked to the 5′ terminus of the nucleotide sequence directing synthesis of the polypeptide having biological activity. Any leader sequence that is functional in a Bacillus host cell of choice may be used in the present invention.

The control sequence may also be a mRNA stabilizing sequence. The term “mRNA stabilizing sequence” is defined herein as a sequence located downstream of a promoter region and upstream of a coding sequence of a polynucleotide encoding a trypsin-like serine protease to which the promoter region is operably linked, such that all mRNAs synthesized from the promoter region may be processed to generate mRNA transcripts with a stabilizer sequence at the 5′ end of the transcripts. For example, the presence of such a stabilizer sequence at the 5′ end of the mRNA transcripts increases their half-life (Agaisse and Lereclus, 1994, supra, Hue et al., 1995, Journal of Bacteriology 177: 3465-3471). The mRNA processing/stabilizing sequence is complementary to the 3′ extremity of bacterial 16S ribosomal RNA. In certain embodiments, the mRNA processing/stabilizing sequence generates essentially single-size transcripts with a stabilizing sequence at the 5′ end of the transcripts. The mRNA processing/stabilizing sequence is preferably one, which is complementary to the 3′ extremity of a bacterial 16S ribosomal RNA. See, U.S. Pat. Nos. 6,255,076 and 5,955,310.

The nucleic acid construct can then be introduced into a Bacillus host cell using methods known in the art or those methods described herein for introducing and expressing a trypsin-like serine protease.

A nucleic acid construct comprising a DNA of interest encoding a protein of interest can also be constructed similarly as described above.

For obtaining secretion of the protein of interest of the introduced DNA, the control sequence may also comprise a signal peptide coding region, which codes for an amino acid sequence linked to the amino terminus of a polypeptide that can direct the expressed polypeptide into the cell's secretory pathway. The signal peptide coding region may be native to the polypeptide or may be obtained from foreign sources. The 5′ end of the coding sequence of the nucleotide sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to that portion of the coding sequence that encodes the secreted polypeptide. The foreign signal peptide coding region may be required where the coding sequence does not normally contain a signal peptide coding region. Alternatively, the foreign signal peptide coding region may simply replace the natural signal peptide coding region in order to obtain enhanced secretion of the polypeptide relative to the natural signal peptide coding region normally associated with the coding sequence. The signal peptide coding region may be obtained from an amylase or a protease gene from a Bacillus species. However, any signal peptide coding region capable of directing the expressed polypeptide into the secretory pathway of a Bacillus host cell of choice may be used in the present invention.

An effective signal peptide coding region for a Bacillus host cell, is the signal peptide coding region obtained from the maltogenic amylase gene from Bacillus NCIB 11837, the Bacillus stearothermophilus alpha-amylase gene, the Bacillus licheniformis subtilisin gene, the Bacillus licheniformis beta-lactamase gene, the Bacillus stearothermophilus neutral proteases genes (nprT, nprS, nprM), and the Bacillus subtilis prsA gene.

Thus, a polynucleotide construct comprising a nucleic acid encoding a trypsin-like serine protease construct comprising a nucleic acid encoding a polypeptide of interest (POI) can be constructed such that it is expressed by a host cell. Because of the known degeneracies in the genetic code, different polynucleotides encoding an identical amino acid sequence can be designed and made with routine skills in the art. For example, codon optimizations can be applied to optimize production in a particular host cell.

Nucleic acids encoding proteins of interest can be incorporated into a vector, wherein the vector can be transferred into a host cell using well-known transformation techniques, such as those disclosed herein.

The vector may be any vector that can be transformed into and replicated within a host cell. For example, a vector comprising a nucleic acid encoding a POI can be transformed and replicated in a bacterial host cell as a means of propagating and amplifying the vector. The vector also may be transformed into a Bacillus expression host of the disclosure, so that the protein encoding nucleic acid (e.g., an ORF) can be expressed as a functional protein.

A representative vector which can be modified with routine skill to comprise and express a nucleic acid encoding a POI is vector p2JM103BBI.

A polynucleotide encoding a trypsin-like serine protease or a POI can be operably linked to a suitable promoter, which allows transcription in the host cell. The promoter may be any nucleic acid sequence that shows transcriptional activity in the host cell of choice and may be derived from genes encoding proteins either homologous or heterologous to the host cell. Means of assessing promoter activity/strength are routine for the skilled artisan.

Examples of suitable promoters for directing the transcription of a polynucleotide sequence encoding comS1 polypeptide or a POI of the disclosure, especially in a bacterial host, include the promoter of the lac operon of E. coli, the Streptomyces coelicolor agarase gene dagA or celA promoters, the promoters of the Bacillus licheniformis alpha-amylase gene (amyl.), the promoters of the Bacillus stearothermophilus maltogenic amylase gene (amyM), the promoters of the Bacillus amyloliquefaciens alpha-amylase (amyQ), the promoters of the Bacillus subtilis xylA and xylB genes, and the like.

A promoter for directing the transcription of a polynucleotide sequence encoding a POI can be a wild-type aprE promoter, a mutant aprE promoter or a consensus aprE promoter set forth in PCT International Publication No. WO2001/51643. In certain other embodiments, a promoter for directing the transcription of a polynucleotide sequence encoding a POI is a wild-type spoVG promoter, a mutant spoVG promoter, or a consensus spoVG promoter (Frisby and Zuber, 1991).

A promoter for directing the transcription of the polynucleotide sequence encoding a trypsin-like serine protease or a POI is a ribosomal promoter such as a ribosomal RNA promoter or a ribosomal protein promoter. The ribosomal RNA promoter can be a rrn promoter derived from B. subtilis, more particularly, the rrn promoter can be a rrnB, rrnI or rrnE ribosomal promoter from B. subtilis. In certain embodiments, the ribosomal RNA promoter is a P2 rrnI promoter from B. subtilis set forth in PCT International Publication No. WO2013/086219.

A suitable vector may further comprise a nucleic acid sequence enabling the vector to replicate in the host cell. Examples of such enabling sequences include the origins of replication of plasmids pUC19, pACYC177, pUB110, pE194, pAMB1, pIJ702, and the like.

A suitable vector may also comprise a selectable marker, e.g., a gene the product of which complements a defect in the isolated host cell, such as the dal genes from B. subtilis or B. licheniformis; or a gene that confers antibiotic resistance such as, e.g., ampicillin resistance, kanamycin resistance, chloramphenicol resistance, tetracycline resistance and the like.

A suitable expression vector typically includes components of a cloning vector, such as, for example, an element that permits autonomous replication of the vector in the selected host organism and one or more phenotypically detectable markers for selection purposes. Expression vectors typically also comprise control nucleotide sequences such as, for example, promoter, operator, ribosome binding site, translation initiation signal and optionally, a repressor gene, one or more activator genes sequences, or the like.

Additionally, a suitable expression vector may further comprise a sequence coding for an amino acid sequence capable of targeting the protein of interest to a host cell organelle such as a peroxisome, or to a particular host cell compartment. Such a targeting sequence may be, for example, the amino acid sequence “SKL”. For expression under the direction of control sequences, the nucleic acid sequence of the protein of interest can be operably linked to the control sequences in a suitable manner such that the expression takes place.

Protocols, such as described herein, used to ligate the DNA construct encoding a protein of interest, promoters, terminators and/or other elements, and to insert them into suitable vectors containing the information necessary for replication, are well known to persons skilled in the art.

An isolated cell, either comprising a polynucleotide construct or an expression vector, is advantageously used as a host cell in the recombinant production of a POI. The cell may be transformed with the DNA construct encoding the POI, conveniently by integrating the construct (in one or more copies) into the host chromosome. Integration is generally deemed an advantage, as the DNA sequence thus introduced is more likely to be stably maintained in the cell. Integration of the DNA constructs into the host chromosome may be performed applying conventional methods, for example, by homologous or heterologous recombination. For example, PCT International Publication No. WO2002/14490 describes methods of Bacillus transformation, transformants thereof and libraries thereof. Alternatively, the cell may be transformed with an expression vector as described above in connection with the different types of host cells.

It is, in other embodiments, advantageous to delete genes from expression hosts, where the gene deficiency can be cured by an expression vector. Known methods may be used to obtain a bacterial host cell having one or more inactivated genes. Gene inactivation may be accomplished by complete or partial deletion, by insertional inactivation or by any other means that renders a gene nonfunctional for its intended purpose, such that the gene is prevented from expression of a functional protein.

Techniques for transformation of bacteria and culturing the bacteria are standard and well known in the art. They can be used to transform the improved hosts of the present invention for the production of recombinant proteins of interest. Introduction of a DNA construct or vector into a host cell includes techniques such as transformation, electroporation, nuclear microinjection, transduction, transfection (e.g., lipofection mediated and DEAE-Dextrin mediated transfection), incubation with calcium phosphate DNA precipitate, high velocity bombardment with DNA-coated microprojectiles, gene gun or biolistic transformation and protoplast fusion, and the like. Transformation and expression methods for bacteria are also disclosed in Brigidi et al. (1990). A general transformation and expression protocol for protease deleted Bacillus strains is described in Ferrari et al. (U.S. Pat. No. 5, 264,366).

Methods for transforming nucleic acids into filamentous fungi such as Aspergillus spp., e.g., A. oryzae or A. niger, H. grisea, H. insolens, and T. reesei. are well known in the art. A suitable procedure for transformation of Aspergillus host cells is described, for example, in EP238023. A suitable procedure for transformation of Trichoderma host cells is described, for example, in Steiger et al 2011, Appl. Environ. Microbiol. 77:114-121.

The choice of a production host can be any suitable microorganism such as bacteria, fungi and algae.

Typically, the choice will depend upon the gene encoding the trypsin-like serine protease and its source.

Introduction of a DNA construct or vector into a host cell includes techniques such as transformation; electroporation; nuclear microinjection; transduction; transfection, (e.g., lipofection mediated and DEAE-Dextrin mediated transfection); incubation with calcium phosphate DNA precipitate; high velocity bombardment with DNA-coated microprojectiles; and protoplast fusion. Basic texts disclosing the general methods that can be used include Sambrook et al., Molecular Cloning, A Laboratory Manual (2nd ed. 1989); Kriegler, Gene Transfer and Expression: A Laboratory Manual (1990); and Ausubel et al., eds., Current Protocols in Molecular Biology (1994)). The methods of transformation of the present invention may result in the stable integration of all or part of the transformation vector into the genome of a host cell, such as a filamentous fungal host cell. However, transformation resulting in the maintenance of a self-replicating extra-chromosomal transformation vector is also contemplated.

Many standard transfection methods can be used to produce bacterial and filamentous fungal (e.g. Aspergillus or Trichoderma) cell lines that express large quantities of the protease. Some of the published methods for the introduction of DNA constructs into cellulase-producing strains of Trichoderma include Lorito, Hayes, DiPietro and Harman, (1993) Curr. Genet. 24: 349-356; Goldman, VanMontagu and Herrera-Estrella, (1990) Curr. Genet. 17:169-174; and Penttila, Nevalainen, Ratto, Salminen and Knowles, (1987) Gene 6: 155-164, also see U.S. Pat. No. 6,022,725; 6,268,328 and Nevalainen et al., “The Molecular Biology of Trichoderma and its Application to the Expression of Both Homologous and Heterologous Genes” in Molecular Industrial Mycology, Eds, Leong and Berka, Marcel Dekker Inc., NY (1992) pp 129-148; for Aspergillus include Yelton, Hamer and Timberlake, (1984) Proc. Natl. Acad. Sci. USA 81: 1470-1474, for Fusarium include Bajar, Podila and Kolattukudy, (1991) Proc. Natl. Acad. Sci. USA 88: 8202-8212, for Streptomyces include Hopwood et al., 1985, Genetic Manipulation of Streptomyces: Laboratory Manual, The John Innes Foundation, Norwich, UK and Fernandez-Abalos et al., Microbiol 149:1623-1632 (2003) and for Bacillus include Brigidi, DeRossi, Bertarini, Riccardi and Matteuzzi, (1990) FEMS Microbiol. Lett. 55: 135-138).

However, any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well-known methods for introducing cloned genomic DNA, cDNA, synthetic DNA or other foreign genetic material into a host cell (see, e.g., Sambrook et al., supra). Also of use is the Agrobacterium-mediated transfection method described in U.S. Pat. No. 6,255,115. It is only necessary that the particular genetic engineering procedure used be capable of successfully introducing at least one gene into the host cell capable of expressing the gene.

After the expression vector is introduced into the cells, the transfected or transformed cells are cultured under conditions favoring expression of genes under control of the promoter sequences.

The medium used to cultivate the cells may be any conventional medium suitable for growing the host cell and obtaining expression of a trypsin-like serine protease polypeptide. Suitable media and media components are available from commercial suppliers or may be prepared according to published recipes (e.g., as described in catalogues of the American Type Culture Collection).

A serine protease polypeptide secreted from the host cells can be used, with minimal post-production processing, as a whole broth preparation.

Depending upon the host cell used post-transcriptional and/or post-translational modifications may be made. One non-limiting example of a post-transcriptional and/or post-translational modification is “clipping” or “truncation” of a polypeptide. For example, this may result in taking an trypsin-like serine protease from an inactive or substantially inactive state to an active state as in the case of a pro-peptide undergoing further post-translational processing to a mature peptide having the enzymatic activity. In another instance, this clipping may result in taking a mature serine protease polypeptide and further removing N or C-terminal amino acids to generate truncated forms of the serine protease that retain enzymatic activity.

Other examples of post-transcriptional or post-translational modifications include, but are not limited to, myristoylation, glycosylation, truncation, lipidation and tyrosine, serine or threonine phosphorylation. The skilled person will appreciate that the type of post-transcriptional or post-translational modifications that a protein may undergo may depend on the host organism in which the protein is expressed.

In some embodiments, the preparation of a spent whole fermentation broth of a recombinant microorganism can be achieved using any cultivation method known in the art resulting in the expression of a trypsin-like serine protease.

Fermentation may, therefore, be understood as comprising shake flask cultivation, small- or large-scale fermentation (including continuous, batch, fed-batch, or solid state fermentations) in laboratory or industrial fermenters performed in a suitable medium and under conditions allowing the trypsin-like serine protease to be expressed or isolated. The term “spent whole fermentation broth” is defined herein as unfractionated contents of fermentation material that includes culture medium, extracellular proteins (e.g., enzymes), and cellular biomass. It is understood that the term “spent whole fermentation broth” also encompasses cellular biomass that has been lysed or permeabilized using methods well known in the art.

Host cells may be cultured under suitable conditions that allow expression of a trypsin-like serine protease. Expression of the enzymes may be constitutive such that they are continually produced, or inducible, requiring a stimulus to initiate expression. In the case of inducible expression, protein production can be initiated when required by, for example, addition of an inducer substance to the culture medium, for example dexamethasone or IPTG or sophorose.

Any of the fermentation methods well known in the art can suitably be used to ferment the transformed or the derivative fungal strain as described above. In some embodiments, fungal cells are grown under batch or continuous fermentation conditions.

A classical batch fermentation is a closed system, where the composition of the medium is set at the beginning of the fermentation, and the composition is not altered during the fermentation. At the beginning of the fermentation, the medium is inoculated with the desired organism(s). In other words, the entire fermentation process takes place without addition of any components to the fermentation system throughout.

Alternatively, a batch fermentation qualifies as a “batch” with respect to the addition of the carbon source. Moreover, attempts are often made to control factors such as pH and oxygen concentration throughout the fermentation process. Typically the metabolite and biomass compositions of the batch system change constantly up to the time the fermentation is stopped. Within batch cultures, cells progress through a static lag phase to a high growth log phase and finally to a stationary phase, where growth rate is diminished or halted. Left untreated, cells in the stationary phase would eventually die. In general, cells in log phase are responsible for the bulk of production of product. A suitable variation on the standard batch system is the “fed-batch fermentation” system. In this variation of a typical batch system, the substrate is added in increments as the fermentation progresses. Fed-batch systems are useful when it is known that catabolite repression would inhibit the metabolism of the cells, and/or where it is desirable to have limited amounts of substrates in the fermentation medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are well known in the art.

Continuous fermentation is another known method of fermentation. It is an open system where a defined fermentation medium is added continuously to a bioreactor, and an equal amount of conditioned medium is removed simultaneously for processing. Continuous fermentation generally maintains the cultures at a constant density, where cells are maintained primarily in log phase growth. Continuous fermentation allows for the modulation of one or more factors that affect cell growth and/or product concentration. For example, a limiting nutrient, such as the carbon source or nitrogen source, can be maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

Separation and concentration techniques are known in the art and conventional methods can be used to prepare a concentrated solution or broth comprising a trypsin-like serine protease polypeptide of the invention. Host cells may be further processed, such as to release enzyme or to improve cell separation, for example by heating or by changing pH or salt content or by treating with enzymes including hen egg white lysozyme, T4 lysozyme, or enzymes described in WO2022047149. For production scale recovery, polypeptides can be enriched or partially purified as generally described above by removing cells via flocculation with polymers. Alternatively, the enzyme can be enriched or purified by microfiltration followed by concentration by ultrafiltration using available membranes and equipment. However, for some applications, the enzyme does not need to be enriched or purified, and whole broth culture can be lysed and used without further treatment. The enzyme can then be processed, for example, into granules.

After fermentation, a fermentation broth is obtained, the microbial cells and various suspended solids, including residual raw fermentation materials, are removed by conventional separation techniques in order to obtain a trypsin-like serine protease solution. Filtration, centrifugation, microfiltration, rotary vacuum drum filtration, ultrafiltration, centrifugation followed by ultra- filtration, extraction, or chromatography, or the like, are generally used.

It may at times be desirable to concentrate a solution or broth comprising a trypsin-like serine protease polypeptide to optimize recovery. Use of un-concentrated solutions or broth would typically increase incubation time in order to collect the enriched or purified enzyme precipitate.

The enzyme-containing solution can be concentrated using conventional concentration techniques until the desired enzyme level is obtained. Concentration of the enzyme containing solution may be achieved by any of the techniques discussed herein. Examples of methods of enrichment and purification include but are not limited to rotary vacuum filtration and/or ultrafiltration.

The trypsin-like serine protease-containing solution or broth may be concentrated until such time the enzyme activity of the concentrated a trypsin-like serine protease polypeptide-containing solution or broth is at a desired level.

Concentration may be performed using, e.g., a precipitation agent, such as a metal halide precipitation agent. Metal halide precipitation agents include but are not limited to alkali metal chlorides, alkali metal bromides and blends of two or more of these metal halides.

Exemplary metal halides include sodium chloride, potassium chloride, sodium bromide, potassium bromide and blends of two or more of these metal halides. The metal halide precipitation agent, sodium chloride, can also be used as a preservative. For production scale recovery, trypsin-like serine protease polypeptides can be enriched or partially purified as generally described above by removing cells via flocculation with polymers. Alternatively, the enzyme can be enriched or purified by microfiltration followed by concentration by ultrafiltration using available membranes and equipment. However, for some applications, the enzyme does not need to be enriched or purified, and whole broth culture can be lysed and used without further treatment. The enzyme can then be processed, for example, into granules.

Trypsin-like serine proteases may be isolated or purified in a variety of ways known to those skilled in the art depending on what other components are present in the sample. Standard purification methods include, but are not limited to, chromatography (e.g., ion exchange, affinity, hydrophobic, chromatofocusing, immunological and size exclusion), electrophoretic procedures (e.g., preparative isoelectric focusing), differential solubility (e.g., ammonium sulfate precipitation), extraction microfiltration, two phase separation. For example, the protein of interest may be purified using a standard anti-protein of interest antibody column. Ultrafiltration and diafiltration techniques, in conjunction with protein concentration, are also useful. For general guidance in suitable purification techniques, see Scopes, Protein Purification (1982). The degree of purification necessary will vary depending on the use of the protein of interest. In some instances, no purification will be necessary.

Assays for detecting and measuring the enzymatic activity of an enzyme, such as a trypsin-like serine protease polypeptide, are well known. Various assays for detecting and measuring activity of proteases (e.g., serine protease polypeptides), are also known to those of ordinary skill in the art. Many well-known procedures exist for measuring proteolytic activity. For example, proteolytic activity may be ascertained by comparative assays that analyze the respective protease's ability to hydrolyze a suitable substrate. Exemplary substrates useful in the analysis of protease or proteolytic activity, include, but are not limited to, di-methyl casein (Sigma C-9801), bovine collagen (Sigma C-9879), bovine elastin (Sigma E-1625), and Keratin Azure (Sigma-Aldrich K8500). Colorimetric assays utilizing these substrates are well known in the art (See e.g., WO99/34011 and U.S. Pat. No. 6,376,450). The pNA peptidyl assay (See e.g., Del Mar et al., Anal Biochem, 99:316-320, 1979) also finds use in determining the active enzyme concentration. This assay measures the rate at which p-nitroaniline is released as the enzyme hydrolyzes a soluble synthetic substrate, such as succinyl-alanine-alanine-proline-phenylalanine-p-nitroanilide (suc-AAPF-pNA). The rate of production of yellow color from the hydrolysis reaction is measured at 405 or 410 nm on a spectrophotometer and is proportional to the active enzyme concentration. In addition, absorbance measurements at 280 nanometers (nm) can be used to determine the total protein concentration in a sample of purified protein. The activity on substrate divided by protein concentration gives the enzyme specific activity.

Some embodiments are directed to a method of cleaning, comprising contacting a surface or an item in need of cleaning with an effective amount of at least one polypeptide described herein or at least one composition described herein; and optionally further comprising the step of rinsing said surface or item after contacting said surface or item with said polypeptide or composition. In other embodiments, the item is dishware or fabric.

Further embodiments are directed to a method of cleaning comprising contacting a surface or an item in need of cleaning with an effective amount of at least one polypeptide having serine protease activity, where the polypeptide is selected from a polypeptide comprising an amino acid sequence with at least 60%, 65%, 70%, 75%, 80%, 85%, 90%, 91%, 92%, 93%, 94%, 95%, 96%, 97%, 98%, 99% or 100% amino acid sequence identity with SEQ ID NOs: 2, 6, 7, 8, and 13; and, optionally, further comprising the step of rinsing said surface or item after contacting said surface or item with the polypeptide.

Still further embodiments are directed to a method of cleaning comprising contacting a surface or an item in need of cleaning with a composition comprising an effective amount of at least one polypeptide having serine protease activity, where the polypeptide is selected from a polypeptide comprising an amino acid sequence with at least 60%, 65%, 70%, 75%, 80%, 85%, 90%, 91%, 92%, 93%, 94%, 95%, 96%, 97%, 98%, 99% or 100% amino acid sequence identity with SEQ ID NOs: 2, 6, 7, 8, and 13; and, optionally, further comprising the step of rinsing said surface or item after contacting said surface or item with the polypeptide.

In still another embodiment, at least one polypeptide described herein has enzymatic activity (e.g., protease activity) and thus is useful in cleaning applications, including but not limited to, methods for cleaning dishware items, tableware items, fabrics, and items having hard surfaces (e.g., the hard surface of a table, table top, wall, furniture item, floor, ceiling, etc.). Some embodiments are directed to at least cleaning composition comprising at least one polypeptide described herein. The enzymatic activity (e.g., protease enzyme activity) of at least one polypeptide described herein can be readily determined through procedures well known to those of ordinary skill in the art. The Examples presented infra describe methods for evaluating cleaning performance. In some embodiments, at least one polypeptide described herein has protease activity in the presence of a surfactant. In other embodiments, the surfactant is selected from the group consisting of a non-ionic surfactant, an anionic surfactant, a cationic surfactant, a zwitterionic surfactant, an ampholytic surfactant, a semi-polar non-ionic surfactant, and a combination thereof. In some embodiments, the protease activity comprises suc-AAPF-pNA activity.

In some embodiments, at least one polypeptide described herein demonstrates cleaning performance in a cleaning composition. Cleaning compositions often include ingredients harmful to the stability and performance of enzymes, making cleaning compositions a harsh environment for enzymes, e.g. serine proteases, to retain function. Thus, it is not trivial for an enzyme to be put in a cleaning composition and expect enzymatic function (e.g. serine protease activity, such as demonstrated by cleaning performance). In some embodiments, one or more serine protease described herein demonstrates cleaning performance in ADW detergent compositions. In some embodiments, the cleaning performance in ADW detergent compositions includes cleaning of egg yolk stains. In some embodiments, one or more serine protease described herein demonstrates cleaning performance in laundry detergent compositions. In some embodiments, the cleaning performance in laundry detergent compositions includes cleaning of blood/milk/ink stains. In one or more cleaning composition described herein, one or more serine protease described herein demonstrates cleaning performance with or without a bleach component.

Unless otherwise noted, all component or composition levels provided herein are made in reference to the active level of that component or composition, and are exclusive of impurities, for example, residual solvents or by-products, which may be present in commercially available sources. Enzyme component weights are based on total active protein. All percentages and ratios are calculated by weight unless otherwise indicated. All percentages and ratios are calculated based on the total composition unless otherwise indicated. Compositions of the invention include detergent compositions. In the exemplified detergent compositions, the enzymes levels are expressed as pure enzyme by weight of the total composition and unless otherwise specified, the detergent ingredients are expressed by weight of the total compositions.

One embodiment is directed to a composition comprising at least one polypeptide having serine protease activity described herein. In some embodiments, the composition is a cleaning composition. In other embodiments, the composition is a detergent composition. In yet other embodiments, the composition is selected from a laundry detergent composition, an ADW detergent composition, a (hand or manual) dishwashing detergent composition, a hard surface cleaning composition, an eyeglass cleaning composition, a medical instrument cleaning composition, a disinfectant (e.g., malodor or microbial) composition, and a personal care cleaning composition. In still other embodiments, the composition is a laundry detergent composition, an ADW deteregent composition, or a (hand or manual) dishwashing detergent composition. Even still further embodiments are directed to a fabric cleaning composition, while other embodiments are directed to a non-fabric cleaning composition.

Some embodiments are directed to a composition described herein, wherein said composition comprises at least one trypsin-like serine protease polypeptide described herein. In other embodiments, the composition described herein comprises at least one polypeptide having serine protease activity, wherein said polypeptide comprises an amino acid sequence with at least 75% identity with the amino acid sequence selected from the group consisting of SEQ ID NOs: 2, 6, 7, 8, and 13. In an even still further embodiment, the composition described herein comprises at least one polypeptide having serine protease activity, wherein said polypeptide comprises an amino acid sequence with at least 80%, 85%, 90%, 91%, 92%, 93%, 94%, 95%, 96%, 97%, 98%, 99%, or 100% identity with the amino acid sequence selected from the group consisting of SEQ ID NOs: 2, 6, 7, 8, and 13. In some embodiments, the composition described herein further comprises one or more surfactant. In some embodiments, the composition described herein further comprises one or more dispersant or dispersing polymers. In yet other embodiments, the at least one trypsin-like serine protease polypeptide having serine protease activity described herein has cleaning activity in one or more composition described herein. In still other embodiments, the at least one polypeptide having serine protease activity described herein has cleaning activity at a temperature between about 16° C. and about 40° C. in one or more composition described herein. In still other embodiments, the composition described herein is selected from a laundry detergent, a fabric softening detergent, a dishwashing detergent, and a hard-surface cleaning detergent.

In some embodiments, a composition described herein further comprises: (i) one or more additional enzymes selected from acyl transferases, amylases, alpha-amylases, beta-amylases, alpha-galactosidases, arabinases, arabinosidases, aryl esterases, beta-galactosidases, beta-glucanases, carrageenases, catalases, chondroitinases, cutinases, endo-beta-mannanases, exo-beta-mannanases, esterases, exo-mannanases, galactanases, glucoamylases, hemicellulases, hexosaminidases, hyaluronidases, keratinases, laccases, lactases, ligninases, lipases, lipolytic enzymes, lipoxygenases, mannanases, metalloproteases, nucleases, oxidases, oxidoreductases, pectate lyases, pectin acetyl esterases, pectinases, pentosanases, perhydrolases, peroxidases, phenoloxidases, phosphatases, phospholipases, phytases, polyesterases, polygalacturonases, additional proteases, pullulanases, reductases, rhamnogalacturonases, cellulases, beta-glucanases, tannases, transglutaminases, xylan acetyl-esterases, xylanases, and xylosidases; and optionally (ii) one or more ions selected from calcium and zinc; (iii) one or more adjunct materials; (iv) one or more stabilizers; (v) one or more bleaching agents; and (vi) combinations thereof.

Another embodiment is directed to a composition comprising one or more adjunct materials and at least one polypeptide described herein. The nature of the adjunct materials employed in any particular composition, and levels of incorporation thereof, will depend on the physical form of the composition and the cleaning application for which such composition will be used.

Exemplary adjunct materials include, but are not limited to, bleach catalysts, an additional enzyme, enzyme stabilizers (including, for example, an enzyme stabilizing system), chelants, optical brighteners, soil release polymers, dye transfer agents, dispersants, suds suppressors, dyes, perfumes, colorants, filler salts, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents, surfactants, builders, chelating agents, dye transfer inhibiting agents, deposition aids, dispersants, additional enzymes, and enzyme stabilizers, catalytic materials, bleach activators, bleach boosters, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, processing aids and/or pigments. Suitable examples of other adjunct materials and levels of use can be found in U.S. Pat. Nos. 5,576,282; 6,306,812; 6,326,348; 6,610,642; 6,605,458; 5,705,464; 5,710,115; 5,698,504; 5,695,679; 5,686,014; and 5,646,101. In embodiments in which one or more adjunct material is not compatible with one or more serine protease described herein suitable methods of keeping the adjunct material(s) and protease(s) separated (i.e., not in contact with each other) can be employed until combination of the two components is appropriate. Such separation methods include any suitable method known in the art (e.g., gelcaps, encapsulation, tablets, physical separation, etc.). The aforementioned adjunct materials may constitute the balance of the cleaning compositions described herein.

In yet another embodiment, at least one composition described herein is

advantageously employed for example, in laundry applications, hard surface cleaning applications, dishwashing applications, including automatic dishwashing and hand dishwashing, as well as cosmetic applications such as dentures, teeth, hair and skin cleaning and disinfecting applications, such as, for example, but not limited to, disinfecting an automatic dishwashing or laundry machine. The at least one polypeptide described herein is also suited for use in contact lens cleaning and wound debridement applications.

In yet still a further embodiment, at least one composition described herein contains phosphate, is phosphate-free, contains boron, is boron-free, or combinations thereof. In other embodiments, the at least one composition described herein is a boron-free composition. In some embodiments, a boron-free composition is a composition to which a borate stabilizer has not been added. In another embodiment, a boron-free composition is a composition that contains less than 5.5% boron. In a still further embodiment, a boron-free composition is a composition that contains less than 4.5% boron. In yet still another embodiment, a boron-free composition is a composition that contains less than 3.5% boron. In yet still a further embodiment, a boron-free composition is a composition that contains less than 2.5% boron. In even further embodiments, a boron-free composition is a composition that contains less than 1.5% boron. In another embodiment, a boron-free composition is a composition that contains less than 1.0% boron. In still further embodiments, a boron-free composition is a composition that contains less than 0.5% boron. In still further embodiments, at least one composition described herein is substantially-free of boron. In some embodiments, at least one composition described herein is phosphate-free. In still other embodiments, at least one composition described herein contains phosphate. In even still other embodiments, at least one composition described herein comprises at least one polypeptide described herein and one or more of an excipient, adjunct material, and/or additional enzyme.

At least one polypeptide described herein also finds use in cleaning additive products. In some embodiments, one or more cleaning additive finds use at low temperatures. Some embodiments provide cleaning additive products comprising at least one polypeptide described herein, which additive is ideally suited for inclusion in a wash process when additional bleaching effectiveness is desired. Such instances include, but are not limited to low temperature cleaning applications. In some embodiments, the additive product is in its simplest form, o at least one polypeptide described herein. In some embodiments, the additive is packaged in dosage form for addition to a cleaning process. In some embodiments, the additive is packaged in dosage form for addition to a cleaning process where a source of peroxygen is employed and increased bleaching effectiveness is desired.

Exemplary fillers or carriers for granular compositions include, but are not limited to, for example, various salts of sulfate, carbonate and silicate; talc; and clay. Exemplary fillers or carriers for liquid compositions include, but are not limited to, for example, water or low molecular weight primary and secondary alcohols including polyols and diols (e.g., methanol, ethanol, propanol and isopropanol). In some embodiments, the compositions contain from about 5% to about 90% of such filler or carrier. Acidic fillers may be included in such compositions to reduce the pH of the resulting solution in the cleaning method or application.

In another embodiment, at least one composition described herein is in a form selected from gel, tablet, powder, granular, solid, liquid, unit dose, and combinations thereof. In yet another embodiment, at least one composition described herein is in a form selected from a low water compact formula, low water HDL or UD, or high water formula or HDL. In some embodiments, the cleaning composition describe herein is in a unit dose form. In other embodiments, the unit does form is selected from pills, tablets, capsules, gelcaps, sachets, pouches, multi-compartment pouches, and pre-measured powders, and liquids. In some embodiments, the unit dose format is designed to provide a controlled release of the ingredients from a multi-compartment pouch (or other unit dose format). Suitable unit dose and controlled release formats are described, for example, in EP2100949; WO 02/102955; U.S. Pat. Nos. 4,765,916; 4,972,017; and WO 04/111178. In some embodiments, the unit dose form is a tablet or powder contained in a water-soluble film or pouch.

The present cleaning compositions or cleaning additives comprise an effective amount of at least one polypeptide described herein, alone or in combination with one or more additional enzyme. Typically, the present cleaning compositions comprise at least about 0.0001 weight percent, from about 0.0001 to about 10, from about 0.001 to about 1, or from about 0.01 to about 0.1 weight percent of at least one trypsin-like serine protease polypeptide described herein. In another embodiment, at least one composition described herein comprises from about 0.01 to about 10 mg, about 0.01 to about 5 mg, about 0.01 to about 2 mg, about 0.01 to about 1 mg, about 0.05 to about 1 mg, about 0.5 to about 10 mg, about 0.5 to about 5 mg, about 0.5 to about 4 mg, about 0.5 to about 4 mg, about 0.5 to about 3 mg, about 0.5 to about 2 mg, about 0.5 to about 1 mg, about 0.1 to about 10 mg, about 0.1 to about 5 mg, about 0.1 to about 4 mg, about 0.1 to about 3 mg, about 0.1 to about 2 mg, about 0.1 to about 2 mg, about 0.1 to about 1 mg, or about 0.1 to about 0.5 mg of at least one polypeptide described herein per gram of composition.

In some embodiments, at least one trypsin-like serine protease polypeptide described herein cleans at low temperatures. In other embodiments, at least one composition described herein cleans at low temperatures. In other embodiments, at least one composition described herein comprises an effective amount of at least one polypeptide described herein as useful or effective for cleaning a surface in need of proteinaceous stain removal.

The compositions described herein are typically formulated such that, during use in aqueous cleaning operations, the wash water will have a pH of from about 4.0 to about 11.5, or even from about 5.0 to about 11.5, or even from about 5.0 to about 8.0, or even from about 7.5 to about 10.5. Liquid product formulations are typically formulated to have a pH from about 3.0 to about 9.0 or even from about 3 to about 5. Granular laundry products are typically formulated to have a pH from about 9 to about 11. Some embodiments provide a composition formulated to have an alkaline pH under wash conditions, such as a pH of from about 8.0 to about 12.0, or from about 8.5 to about 11.0, or from about 9.0 to about 11.0. In some embodiments, the composition described herein is formulated to have a neutral pH under wash conditions, such as a pH of from about 5.0 to about 8.0, or from about 5.5 to about 8.0, or from about 6.0 to about 8.0, or from about 6.0 to about 7.5. In some embodiments, the neutral pH conditions can be measured when the composition is dissolved 1:100 (wt:wt) in de-ionised water at 20° C. and measured using a conventional pH meter. Techniques for controlling pH include the use of buffers, alkalis, acids, etc., and are well known to those skilled in the art.

In some embodiments, when the at least one polypeptide described herein is employed in a granular composition or liquid, it is desirable for the polypeptide to be in the form of an encapsulated particle to protect it from other components in the composition during storage. In addition, encapsulation is also a means of controlling the availability of the polypeptide during the cleaning process. In some embodiments, encapsulation enhances the performance of polypeptide and/or additional enzymes. In this regard, at least one polypeptide described herein is encapsulated with any suitable encapsulating material known in the art. In some embodiments, the encapsulating material typically encapsulates at least part of the polypeptide. Typically, the encapsulating material is water-soluble and/or water-dispersible. In some embodiments, the encapsulating material has a glass transition temperature (Tg) of 0° C. or higher. Tg is described in more detail in WO97/11151. The encapsulating material is typically selected from carbohydrates, natural or synthetic gums, chitin, chitosan, cellulose and cellulose derivatives, silicates, phosphates, borates, polyvinyl alcohol, polyethylene glycol, paraffin waxes, and combinations thereof. When the encapsulating material is a carbohydrate, it is typically selected from monosaccharides, oligosaccharides, polysaccharides, and combinations thereof. In some typical embodiments, the encapsulating material is a starch (See e.g.,

EP0922499; U.S. Pat. Nos. 4,977,252; 5,354,559, and 5,935,826). In some embodiments, the encapsulating material is a microsphere made from plastic such as thermoplastics, acrylonitrile, methacrylonitrile, polyacrylonitrile, polymethacrylonitrile and mixtures thereof; commercially available microspheres that find use include, but are not limited to those supplied by EXPANCEL® (Akzo Nobel Chemicals International, B.V.), and PM6545, PM6550, PM7220, PM7228, EXTENDOSPHERESR (Sphere One Inc.), LUXSILR (Potters Industries LLC), Q-CEL® (Potters Industries LLC), and SPHERICEL R (Potters Industries LLC).

There are a variety of wash conditions including varying detergent formulations, wash water volumes, wash water temperatures, and lengths of wash time, to which proteases involved in washing are exposed. A low detergent concentration system includes detergents where less than about 800 ppm of the detergent components are present in the wash water. A medium detergent concentration includes detergents where between about 800 ppm and about 2000ppm of the detergent components are present in the wash water. A high detergent concentration system includes detergents where greater than about 2000 ppm of the detergent components are present in the wash water. In some embodiments, the “cold water washing” of the present invention utilizes “cold water detergent” suitable for washing at temperatures from about 10° ° C. to about 40° C., or from about 20° C. to about 30° C., or from about 15° C. to about 25° C., as well as all other combinations within the range of about 15° C. to about 35° C., and all ranges within 10° C. to 40° C.

Different geographies typically have different water hardness. Water hardness is usually described in terms of the grains per gallon mixed Ca²⁺/Mg²⁺. Hardness is a measure of the amount of calcium (Ca²⁺) and magnesium (Mg²⁺) in the water. Most water in the United States is hard, but the degree of hardness varies. Moderately hard (60-120 ppm) to hard (121-181 ppm) water has 60 to 181 parts per million.

| Water | Grains per gallon | Parts per million |
|---|---|---|
| Soft | less than 1.0 | less than 17 |
| Slightly hard | 1.0 to 3.5 | 17 to 60 |
| Moderately hard | 3.5 to 7.0 | 60 to 120 |
| Hard | 7.0 to 10.5 | 120 to 180 |
| Very hard | greater than 10.5 | greater than 180 |

European water hardness is typically greater than about 10.5 (for example about 10.5 to about 20.0) grains per gallon mixed Ca²⁺/Mg²⁺ (e.g., about 15 grains per gallon mixed Ca²⁺/Mg²⁺). North American water hardness is typically greater than Japanese water hardness, but less than European water hardness. For example, North American water hardness can be between about 3 to about 10 grains, about 3 to about 8 grains or about 6 grains. Japanese water hardness is typically lower than North American water hardness, usually less than about 4, for example about 3 grains per gallon mixed Ca²⁺/Mg²⁺.

Other embodiments are directed to at least one composition comprising from about 0.00001% to about 10% by weight composition of at least one polypeptide described herein and from about 99.999% to about 90.0% by weight composition of one or more adjunct material. In another embodiment, the composition comprises from about 0.0001% to about 10%, about 0.001% to about 5%, about 0.001% to about 2%, or about 0.005% to about 0.5% by weight composition of at least one polypeptide described herein and from about 99.9999% to about 90.0%, about 99.999% to about 98%, about 99.995% to about 99.5% by weight composition of one or more adjunct material.

In other embodiments, the composition described herein comprises at least one polypeptide described herein and one or more additional enzymes. The one or more additional enzyme is selected from acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1, 4-glucanases, endo-beta-mannanases, esterases, exo-mannanases, galactanases, glucoamylases, hemicellulases, hexosaminidases, hyaluronidases, keratinases, laccases, lactases, ligninases, lipases, lipoxygenases, malanases, mannanases, metalloproteases, nucleases, oxidases, pectate lyases, pectin acetyl esterases, pectinases, pentosanases, peroxidases, phenoloxidases, phosphatases, phospholipases, phytases, polygalacturonases, additional proteases, pullulanases, reductases, rhamnogalacturonases, beta-glucanases, tannases, transglutaminases, xylan acetyl-esterases, xylanases, xyloglucanases, xylosidases, and any combination or mixture thereof. Some embodiments are directed to a combination of enzymes (i.e., a “cocktail”) comprising conventional enzymes like amylase, lipase, cutinase and/or cellulase in conjunction with at least one polypeptide described herein and/or one or more additional protease.

In another embodiment, a composition as provided herein comprises one or more trypsin-like serine protease described herein and one or more additional protease. In one embodiment, the additional protease is a serine protease. In another embodiment, the additional protease is a metalloprotease, a fungal subtilisin, or an alkaline microbial protease or a second trypsin-like protease. Suitable additional proteases include those of animal, vegetable or microbial origin. In some embodiments, the additional protease is a microbial protease. In other embodiments, the additional protease is a chemically or genetically modified mutant. In another embodiment, the additional protease is an alkaline microbial protease or a second trypsin-like protease. In other embodiments, the additional protease does not contain cross-reactive epitopes with the trypsin-like serine protease as measured by antibody binding or other assays available in the art. Exemplary alkaline proteases include subtilisins derived from, for example, Bacillus (e.g., BPN', Carlsberg, subtilisin 309, subtilisin 147, B. gibsonii, B. pumilus DSM18097, and subtilisin 168), or fungal origin, such as, for example, those described in U.S. Pat. No. 8,362,222. Exemplary additional proteases include but are not limited to those described in WO92/21760, WO95/23221, WO2008/010925, WO09/149200, WO09/149144, WO09/149145, WO 10/056640, WO10/056653, WO2010/0566356, WO11/072099, WO2011/13022, WO11/140364, WO 12/151534, WO2015/038792, WO2015/089447, WO2015/089441, WO 2017/215925, US Publ. No. 2008/0090747, U.S. Pat. Nos. 5,801,039, 5,340,735, 5,500,364, 5,855,625, RE 34,606, U.S. Pat. Nos. 5,955,340, 5,700,676 6,312,936, 6,482,628, 8,530,219, US Provisional Appl Nos. 62/180673 and 62/161077, and PCT Appl Nos. PCT/US2015/021813, PCT/US2015/055900, PCT/US2015/057497, PCT/US2015/057492, PCT/US2015/057512, PCT/US2015/057526, PCT/US2015/057520, PCT/US2015/057502, PCT/US2016/022282, and PCT/US16/32514, as well as metalloproteases described in WO1999014341, WO1999033960, WO1999014342, WO1999034003, WO2007044993, WO2009058303, WO 2009058661, WO2014071410, WO2014194032, WO2014194034, WO 2014194054, WO 2014/194117, EP3380599, WO2017215925, and WO2016203064. Exemplary additional proteases include, but are not limited to trypsin (e.g., of porcine or bovine origin) and the Fusarium protease described in WO89/06270. Exemplary commercial proteases include, but are not limited to MAXATASE™, MAXACAL™, MAXAPEM™, OPTICLEAN®, OPTIMASE®, PROPERASE®, PURAFECT®, PURAFECT® OXP, PURAMAX™, EXCELLASE™, PREFERENZ™ proteases (e.g. P100, P110, P280, P300), EFFECTENZ™ proteases (e.g. P1000, P1050, P2000), EXCELLENZ™ proteases (e.g. P1000), ULTIMASE®, and PURAFAST™ (DuPont/Danisco/Genencor); ALCALASE®, ALCALASE®, ULTRA, BLAZE®, BLAZE® variants, BLAZE® EVITY®, BLAZE® EVITY® 16L, CORONASE®, SAVINASE®, SAVINASE® ULTRA, SAVINASE® EVITY®, SAVINASE® EVERIS®, PRIMASE®, DURAZYM™, POLARZYME®, OVOZYME®, KANNASE®, LIQUANASE®, LIQUANASE EVERIS®, NEUTRASE®, PROGRESS UNO®, RELASE®, and ESPERASE® (Novozymes); BLAP™ and BLAP™ variants (Henkel); LAVERGY™ PRO 104 L, PRO 106 LS, PRO 114 LS (BASF), KAP (B. alkalophilus subtilisin (Kao)) and BIOTOUCH® (AB Enzymes).

Another embodiment is directed to a composition comprising at least one polypeptide described herein and one or more lipase. In some embodiments, the composition comprises from about 0.00001% to about 10%, about 0.0001% to about 10%, about 0.001% to about 5%, about 0.001% to about 2%, or about 0.005% to about 0.5% lipase by weight composition. An exemplary lipase can be a chemically or genetically modified mutant. Exemplary lipases include, but are not limited to, e.g., those of bacterial or fungal origin, such as, e.g., H. lanuginosa lipase (see, e.g., EP 258068 and EP 305216), T. lanuginosus lipase (see, e.g., WO 2014/059360 and WO2015/010009), Rhizomucor miehei lipase (see, e.g., EP 238023), Candida lipase, such as (C. antarctica lipase (e.g., C. antarctica lipase A or B) (see, e.g., EP 214761), Pseudomonas lipases such as P. alcaligenes and P. pseudoalcaligenes lipase (see, e.g., EP 218272), P. cepacia lipase (see, e.g., EP 331376), P. stutzeri lipase (see, e.g., GB 1,372,034), P. fluorescens lipase, Bacillus lipase (e.g., B. subtilis lipase (Dartois et al., Biochem. Biophys. Acta 1131:253-260 (1993)), B. stearothermophilus lipase (see, e.g., JP 64/744992), and B. pumilus lipase (see, e.g., WO 91/16422)). Exemplary cloned lipases include, but not limited to Penicillium camembertii lipase (See, Yamaguchi et al., Gene 103:61-67 (1991)), Geotricum candidum lipase (See, Schimada et al., J. Biochem., 106:383-388 (1989)), and various Rhizopus lipases, such as, R. delemar lipase (See, Hass et al., Gene 109:117-113 (1991)), R. niveus lipase (Kugimiya et al., Biosci. Biotech. Biochem. 56:716-719 (1992)) and R. oryzae lipase. Other lipolytic enzymes, such as cutinases, may also find use in one or more composition describe herein, including, but not limited to, e.g., cutinase derived from Pseudomonas mendocina (see, WO 88/09367) and/or Fusarium solani pisi (see, WO90/09446). Exemplary commercial lipases include, but are not limited to M1 LIPASE, LUMA FAST, and LIPOMAX (Genecor); LIPEX®, LIPOCLEAN®, LIPOLASE® and LIPOLASE® ULTRA (Novozymes); and LIPASE PS (Amano Pharmaceutical Co. Ltd).

A still further embodiment is directed to a composition comprising at least one trypsin-like serine protease polypeptide described herein and one or more amylase. In one embodiment, the composition comprises from about 0.00001% to about 10%, about 0.0001% to about 10%, about 0.001% to about 5%, about 0.001% to about 2%, or about 0.005% to about 0.5% amylase by weight composition. Any amylase (e.g., alpha and/or beta) suitable for use in alkaline solutions may be useful to include in such composition. An exemplary amylase can be a chemically or genetically modified mutant. Exemplary amylases include, but are not limited to those of bacterial or fungal origin, such as, for example, amylases described in GB 1,296,839, WO9100353, WO9402597, WO94183314, WO9510603, WO9526397, WO9535382, WO9605295, WO9623873, WO9623874, WO 9630481, WO9710342, WO9741213, WO9743424, WO9813481, WO 9826078, WO9902702, WO 9909183, WO9919467, WO9923211, WO9929876, WO9942567, WO 9943793, WO9943794, WO 9946399, WO0029560, WO0060058, WO0060059, WO0060060, WO 0114532, WO0134784, WO 0164852, WO0166712, WO0188107, WO0196537, WO02092797, WO 0210355, WO0231124, WO 2004055178, WO2004113551, WO2005001064, WO2005003311, WO 2005018336, WO2005019443, WO2005066338, WO2006002643, WO2006012899, WO2006012902, WO2006031554, WO 2006063594, WO2006066594, WO2006066596, WO2006136161, WO 2008000825, WO2008088493, WO2008092919, WO2008101894, WO2008/112459, WO2009061380, WO2009061381, WO 2009100102, WO2009140504, WO2009149419, WO 2010059413, WO 2010088447, WO2010091221, WO2010104675, WO2010115021, WO10115028, WO2010117511, WO 2011076123, WO2011076897, WO2011080352, WO2011080353, WO 2011080354, WO2011082425, WO2011082429, WO 2011087836, WO2011098531, WO2013063460, WO2013184577, WO 2014099523, WO2014164777, WO2015077126, and WO2016203064. Exemplary commercial amylases include, but are not limited to AMPLIFY®, AMPLIFY PRIME®, BAN, DURAMYL®, TERMAMYL®, TERMAMYL® ULTRA, FUNGAMYL®, STAINZYME®, STAINZYME® PLUS, STAINZYME® ULTRA, and STAINZYME® EVITY® (Novozymes); EFFECTENZ™ S 1000, POWERASE®, PREFERENZ™ S 100, PREFERENZ™ S 110, EXCELLENZ™ S 2000, RAPIDASE® and MAXAMYL® P (Danisco US).

Yet a still further embodiment is directed to a composition comprising at least one polypeptide described herein and one or more cellulase. In one embodiment, the composition comprises from about 0.00001% to about 10%, 0.0001% to about 10%, about 0.001% to about 5%, about 0.001% to about 2%, or about 0.005% to about 0.5% cellulase by weight of composition. Any suitable cellulase may find used in a composition described herein. An exemplary cellulase can be a chemically or genetically modified mutant. Exemplary cellulases include but are not limited, to those of bacterial or fungal origin, such as, for example, is described in WO2005054475, WO2005056787, U.S. Pat. No. 7,449,318, 7,833,773, 4,435,307; EP 0495257; and US Provisional Appl. No. 62/296,678. Exemplary commercial cellulases include, but are not limited to, CELLUCLEAN®, CELLUZYME®, CAREZYME®, ENDOLASE®, RENOZYME®, and CAREZYME® PREMIUM (Novozymes); REVITALENZ® 100, REVITALENZ® 200/220, and REVITALENZ® 2000 (Danisco US); and KAC-500(B) (Kao Corporation). In some embodiments, cellulases are incorporated as portions or fragments of mature wild-type or variant cellulases, wherein a portion of the N-terminus is deleted (see, e.g., U.S. Pat. No. 5,874,276).

An even still further embodiment is directed to a composition comprising at least one polypeptide described herein and one or more mannanase. In one embodiment, the composition comprises from about 0.00001% to about 10%, about 0.0001% to about 10%, about 0.001% to about 5%, about 0.001% to about 2%, or about 0.005% to about 0.5% mannanase by weight composition. An exemplary mannanase can be a chemically or genetically modified mutant. Exemplary mannanases include, but are not limited to, those of bacterial or fungal origin, such as, for example, as is described in WO2016007929, U.S. Pat. Nos. 6,566,114, 6,602,842, and 6,440,991, and International Appl Nos. PCT/US2016/060850 and PCT/US2016/060844. Exemplary commercial mannanases include, but are not limited to MANNAWAYR (Novozymes) and EFFECTENZ™ M 1000, EFFECTENZ™ M 2000, PREFERENZ® M 100, MANNASTAR®, and PURABRITE (Danisco US).

A yet even still further embodiment is directed to a composition comprising at least one polypeptide described herein and one or more peroxidase and/or oxidase enzyme. In one embodiment, the composition comprises from about 0.00001% to about 10%, about 0.0001% to about 10%, about 0.001% to about 5%, about 0.001% to about 2%, or about 0.005% to about 0.5% peroxidase or oxidase by weight composition. A peroxidase may be used in combination with hydrogen peroxide or a source thereof (e.g., a percarbonate, perborate or persulfate) and an oxidase may be used in combination with oxygen. Peroxidases and oxidases are used for “solution bleaching” (i.e., to prevent transfer of a textile dye from a dyed fabric to another fabric when the fabrics are washed together in a wash liquor), alone or in combination with an enhancing agent (see, e.g., WO9412621 and WO9501426). An exemplary peroxidase and/or oxidase can be a chemically or genetically modified mutant. Exemplary peroxidases/oxidases include, but are not limited to those of plant, bacterial, or fungal origin.

Another embodiment is directed to a composition comprising at least one polypeptide described herein and one or more perhydrolase, such as, for example, is described in WO2005056782, WO2007106293, WO2008063400, WO2008106214, and WO2008106215.

In yet another embodiment, at least one polypeptide described herein and one or more additional enzyme contained in at least one composition described herein may each independently range to about 10%, wherein the balance of the cleaning composition is one or more adjunct material.

In some embodiments, at least one composition described herein finds use as a detergent additive, wherein said additive is in a solid or liquid form. Such additive products are intended to supplement and/or boost the performance of conventional detergent compositions and can be added at any stage of the cleaning process. In some embodiments, the density of the laundry detergent composition ranges from about 400 to about 1200 g/liter, while in other embodiments it ranges from about 500 to about 950 g/liter of composition measured at 20° C.

Some embodiments are directed to a laundry detergent composition comprising at least one polypeptide described herein and one or more adjunct materials selected from surfactants, enzyme stabilizers, builder compounds, polymeric compounds, bleaching agents, additional enzymes, suds suppressors, dispersants, lime-soap dispersants, soil suspension agents, anti-redeposition agents, corrosion inhibitors, and combinations thereof. In some embodiments, the laundry compositions also contain softening agents.

Further embodiments are directed to manual dishwashing compositions comprising at least one polypeptide described herein and one or more adjunct material selected from surfactants, organic polymeric compounds, suds enhancing agents, group II metal ions, solvents, hydrotropes, and additional enzymes.

Other embodiments are directed to at least one composition described herein, wherein said composition is a compact granular fabric cleaning composition that finds use in laundering colored fabrics or provides softening through the wash capacity, or is a heavy duty liquid (HDL) fabric cleaning composition. Exemplary fabric cleaning compositions and/or processes for making are described in U.S. Pat. Nos. 6,610,642 and 6,376,450. Other exemplary cleaning compositions are described, for example, in U.S. Pat. Nos. 6,605,458; 6,294,514; 5,929,022; 5,879,584; 5,691,297; 5,565,145; 5,574,005; 5,569,645; 5,565,422; 5,516,448; 5,489,392; and 5,486,303; 4,968,451; 4,597,898; 4,561,998; 4,550,862; 4,537,706; 4,515,707; and 4,515,705.

In some embodiments, the cleaning compositions comprise an acidifying particle or an amino carboxylic builder. Examples of an amino carboxylic builder include aminocarboxylic acids, salts and derivatives thereof. In some embodiment, the amino carboxylic builder is an aminopolycarboxylic builder, such as glycine-N,N-diacetic acid or derivative of general formula MOOC—CHR—N(CH₂COOM)₂ where R is C₁₋₁₂alkyl and M is alkali metal. In some embodiments, the amino carboxylic builder can be methylglycine diacetic acid (MGDA), GLDA (glutamic-N,N-diacetic acid), iminodisuccinic acid (IDS), carboxymethyl inulin and salts and derivatives thereof, aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl) aspartic acid (SMAS), N-(2-sulfoethyl)aspartic acid (SEAS), N-(2-sulfomethyl)glutamic acid (SMGL), N-(2-sulfoethyl) glutamic acid (SEGL), IDS (iminodiacetic acid) and salts and derivatives thereof such as N-methyliminodiacetic acid (MIDA), alpha-alanine-N,N-diacetic acid (alpha-ALDA), serine-N,N-diacetic acid (SEDA), isoserine-N,Ndiacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA) and alkali metal salts and derivative thereof. In some embodiments, the acidifying particle has a weight geometric mean particle size of from about 400μ to about 1200μ and a bulk density of at least 550 g/L. In some embodiments, the acidifying particle comprises at least about 5% of the builder.

In some embodiments, the acidifying particle can comprise any acid, including organic acids and mineral acids. Organic acids can have one or two carboxyls and in some instances up to 15 carbons, especially up to 10 carbons, such as formic, acetic, propionic, capric, oxalic, succinic, adipic, maleic, fumaric, sebacic, malic, lactic, glycolic, tartaric and glyoxylic acids. In some embodiments, the acid is citric acid. Mineral acids include hydrochloric and sulphuric acid. In some instances, the acidifying particle is a highly active particle comprising a high level of amino carboxylic builder. Sulphuric acid has also been found to further contribute to the stability of the final particle.

Additional embodiments are directed to a cleaning composition comprising at least one polypeptide described herein and one or more surfactant and/or surfactant system, wherein the surfactant is selected from nonionic surfactants, anionic surfactants, cationic surfactants, ampholytic surfactants, zwitterionic surfactants, semi-polar nonionic surfactants, and mixtures thereof. In one embodiment, the surfactant is a nonionic alcohol ethoxylate. In some embodiments, the surfactant is present at a level of from about 0.1 to about 60%, while in alternative embodiments the level is from about 1 to about 50%, while in still further embodiments the level is from about 5 to about 40%, by weight of the composition.

In some embodiments, at least one composition described herein comprises one or more detergent builders or builder systems. In one embodiment, the composition comprises from about 1%, from about 0.1% to about 80%, from about 3% to about 60%, from about 5% to about 40%, or from about 10% to about 50% builder by weight composition. Exemplary builders include, but are not limited to alkali metal; ammonium and alkanolammonium salts of polyphosphates; alkali metal silicates; alkaline earth and alkali metal carbonates; aluminosilicates; polycarboxylate compounds; ether hydroxypolycarboxylates; copolymers of maleic anhydride with ethylene or vinyl methyl ether, 1,3,5-trihydroxy benzene-2,4,6-trisulphonic acid, and carboxymethyloxysuccinic acid; ammonium and substituted ammonium salts of polyacetic acids such as ethylenediamine tetraacetic acid and nitrilotriacetic acid; polycarboxylates such as mellitic acid, succinic acid, citric acid, oxydisuccinic acid, polymaleic acid, benzene 1,3,5-tricarboxylic acid, carboxymethyloxysuccinic acid; and soluble salts thereof. In some such compositions, the builders form water-soluble hardness ion complexes (e.g., sequestering builders), such as citrates and polyphosphates, e.g., sodium tripolyphosphate, sodium tripolyphospate hexahydrate, potassium tripolyphosphate, and mixed sodium and potassium tripolyphosphate. Exemplary builders are described in, e.g., EP 2100949. In some embodiments, the builders include phosphate builders and non-phosphate builders. In some embodiments, the builder is a phosphate builder. In some embodiments, the builder is a non-phosphate builder. In some embodiments, the builder comprises a mixture of phosphate and non-phosphate builders. Exemplary phosphate builders include, but are not limited to mono-phosphates, di-phosphates, tri-polyphosphates or oligomeric-poylphosphates, including the alkali metal salts of these compounds, including the sodium salts. In some embodiments, a builder can be sodium tripolyphosphate (STPP). Additionally, the composition can comprise carbonate and/or citrate. Other suitable non-phosphate builders include homopolymers and copolymers of polycarboxylic acids and their partially or completely neutralized salts, monomeric polycarboxylic acids and hydroxycarboxylic acids and their salts. In some embodiments, salts of the above mentioned compounds include the ammonium and/or alkali metal salts, i.e. the lithium, sodium, and potassium salts, including sodium salts. Suitable polycarboxylic acids include acyclic, alicyclic, hetero-cyclic and aromatic carboxylic acids, wherein in some embodiments, they can contain at least two carboxyl groups which are in each case separated from one another by, in some instances, no more than two carbon atoms.

In some embodiments, at least one composition described herein comprises one or more chelating agent. In one embodiment, the composition comprises from about 0.1% to about 15% or about 3% to about 10% chelating agent by weight composition. Exemplary chelating agents include, but are not limited to, e.g., copper, iron, manganese, and mixtures thereof.

In some embodiments, at least one composition described herein comprises one or more deposition aid. Exemplary deposition aids include, but are not limited to, e.g., polyethylene glycol; polypropylene glycol; polycarboxylate; soil release polymers, such as, e.g., polytelephthalic acid; clays such as, e.g., kaolinite, montmorillonite, atapulgite, illite, bentonite, and halloysite; and mixtures thereof.

In other embodiments, at least one composition described herein comprises one or more anti-redeposition agent or non-ionic surfactant (which can prevent the re-deposition of soils) (see, e.g., EP 2100949). For example, in ADW compositions, non-ionic surfactants find use for surface modification purposes, in particular for sheeting, to avoid filming and spotting and to improve shine. These non-ionic surfactants also find use in preventing the re-deposition of soils. In some embodiments, the non-ionic surfactant can be ethoxylated nonionic surfactants, epoxy-capped poly(oxyalkylated) alcohols and amine oxides surfactants.

In some embodiments, at least one composition described herein comprises one or more dye transfer inhibiting agent. Exemplary polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones, polyvinylimidazoles, and mixtures thereof. In one embodiment, the composition comprises from about 0.0001% to about 10%, about 0.01% to about 5%, or about 0.1% to about 3% dye transfer inhibiting agent by weight composition.

In some embodiments, at least one composition described herein comprises one or more silicate. Exemplary silicates include, but are not limited to, sodium silicates, e.g., sodium disilicate, sodium metasilicate, and crystalline phyllosilicates. In some embodiments, silicates are present at a level of from about 1% to about 20% or about 5% to about 15% by weight of the composition.

In some additional embodiments, at least one composition described herein comprises one or more dispersant or dispersant polymer. Exemplary water-soluble organic materials include, but are not limited to, e.g., homo- or co-polymeric acids or their salts, in which the polycarboxylic acid comprises at least two carboxyl radicals separated from each other by not more than two carbon atoms. A dispersant polymer can be used in any suitable amount from about 0.1 to about 20%, preferably from 0.2 to about 15%, more preferably from 0.3 to % by weight of the composition.

In some further embodiments, at least one composition described herein comprises one or more enzyme stabilizer. In some embodiments, the enzyme stabilizer is water-soluble sources of calcium and/or magnesium ions. In some embodiments, the enzyme stabilizers include oligosaccharides, polysaccharides, and inorganic divalent metal salts, including alkaline earth metals, such as calcium salts. In some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (III), tin (II), cobalt (II), copper (II), nickel (II), and oxovanadium (IV)). Chlorides and sulfates also find use in some embodiments. Exemplary oligosaccharides and polysaccharides (e.g., dextrins) are described, for example, in WO 07/145964. In some embodiments, reversible protease inhibitors also find use, such as boron-containing compounds (e.g., borate, 4-formyl phenyl boronic acid, and phenyl-boronic acid derivatives (such as for example, those described in WO96/41859)) and/or a peptide aldehyde, such as, for example, is further described in WO2009/118375 and WO2013004636.

Peptide aldehydes may be used as protease stabilizers in detergent formulations as previously described (WO199813458, WO2011036153, US20140228274). Examples of peptide aldehyde stabilizers are peptide aldehydes, ketones, or halomethyl ketones and might be ‘N-capped’ with for instance a ureido, a carbamate, or a urea moiety, or ‘doubly N-capped’ with for instance a carbonyl, a ureido, an oxiamide, a thioureido, a dithiooxamide, or a thiooxamide moiety (EP2358857B1). The molar ratio of these inhibitors to the protease may be 0.1:1 to 100:1, e.g. 0.5:1-50:1, 1:1-25:1 or 2:1-10:1. Other examples of protease stabilizers are benzophenone or benzoic acid anilide derivatives, which might contain carboxyl groups (U.S. Pat. No. 7,968,508 B2). The molar ratio of these stabilizers to protease is preferably in the range of 1:1 to 1000:1 in particular 1:1 to 500:1 especially preferably from 1:1 to 100:1, most especially preferably from 1:1 to 20:1.

In some embodiments, at least one composition described herein comprises one or more bleach, bleach activator, and/or bleach catalyst. In some embodiments, at least one composition described herein comprises one or more inorganic and/or organic bleaching compound. Exemplary inorganic bleaches include, but are not limited to perhydrate salts, e.g., perborate, percarbonate, perphosphate, persulfate, and persilicate salts. In some embodiments, inorganic perhydrate salts are alkali metal salts. In some embodiments, inorganic perhydrate salts are included as the crystalline solid, without additional protection, although in some other embodiments, the salt is coated. Bleach activators are typically organic peracid precursors that enhance the bleaching action in the course of cleaning at temperatures of 60° C. and below. Exemplary bleach activators include compounds which, under perhydrolysis conditions, give aliphatic peroxoycarboxylic acids having from about 1 to about 10 carbon atoms or about 2 to about 4 carbon atoms, and/or optionally substituted perbenzoic acid. Exemplary bleach activators ae described, for example, in EP 2100949. Exemplary bleach catalysts include, but are not limited to, manganese triazacyclononane and related complexes, as well as cobalt, copper, manganese, and iron complexes. Additional exemplary bleach catalysts are described, for example, in U.S. Pat. Nos 4,246,612; 5,227,084; 4,810,410; WO 99/06521; and EP 2100949.

In some embodiments, at least one composition described herein comprises one or more catalytic metal complexes. In some embodiments, a metal-containing bleach catalyst finds use. In some embodiments, the metal bleach catalyst comprises a catalyst system comprising a transition metal cation of defined bleach catalytic activity (e.g., copper, iron, titanium, ruthenium, tungsten, molybdenum, or manganese cations), an auxiliary metal cation having little or no bleach catalytic activity (e.g., zinc or aluminum cations), and a sequestrate having defined stability constants for the catalytic and auxiliary metal cations, particularly ethylenediaminetetraacetic acid, ethylenediaminetetra (methylenephosphonic acid) and water-soluble salts thereof (see, e.g., U.S. Pat. No. 4,430,243). In some embodiments, one or more composition described herein is catalyzed by means of a manganese compound. Such compounds and levels of use are described, for example, in U.S. Pat. No. 5,576,282. In additional embodiments, cobalt bleach catalysts find use and are included in one or more composition described herein. Various cobalt bleach catalysts are described, for example, in U.S. Pat. Nos. 5,597,936 and 5,595,967.

In some additional embodiments, at least one described herein includes a transition metal complex of a macropolycyclic rigid ligand (MRL). As a practical matter, and not by way of limitation, in some embodiments, the compositions and cleaning processes described herein are adjusted to provide on the order of at least one part per hundred million, from about 0.005 ppm to about 25 ppm, about 0.05 ppm to about 10 ppm, or about 0.1 ppm to about 5 ppm of active MRL in the wash liquor. Exemplary MRLs include, but are not limited to special ultra-rigid ligands that are cross-bridged, such as, e.g., 5,12-diethyl-1,5,8,12-tetraazabicyclo (6.6.2) hexadecane. Exemplary metal MRLs are described, for example, in WO 2000/32601 and U.S. Pat. No. 6,225,464.

In another embodiment, at least one composition described herein comprises one or more metal care agent. In some embodiments, the composition comprises from about 0.1% to about 5% metal care agent by weight composition. Exemplary metal care agents include, for example, aluminum, stainless steel, and non-ferrous metals (e.g., silver and copper). Additional exemplary metal care agents are described, for example, in EP 2100949, WO 94/26860, and WO 94/26859. In some compositions, the metal care agent is a zinc salt.

In some embodiments, the cleaning composition is a high density liquid (HDL) composition comprising at least one polypeptide described herein. The HDL liquid laundry detergent can comprise a detersive surfactant (10-40%) comprising anionic detersive surfactant selected from a group of linear or branched or random chain, substituted or unsubstituted alkyl sulphates, alkyl sulphonates, alkyl alkoxylated sulphate, alkyl phosphates, alkyl phosphonates, alkyl carboxylates, and/or mixtures thereof; and optionally non-ionic surfactant selected from a group of linear or branched or random chain, substituted or unsubstituted alkyl alkoxylated alcohol, for example, a C₈-C₁₈alkyl ethoxylated alcohol and/or C₆-C₁₂alkyl phenol alkoxylates, optionally wherein the weight ratio of anionic detersive surfactant (with a hydrophilic index (HIc) of from 6.0 to 9) to non-ionic detersive surfactant is greater than 1:1. Suitable detersive surfactants also include cationic detersive surfactants (selected from alkyl pyridinium compounds, alkyl quarternary ammonium compounds, alkyl quarternary phosphonium compounds, alkyl ternary sulphonium compounds, and/or mixtures thereof); zwitterionic and/or amphoteric detersive surfactants (selected from alkanolamine sulpho-betaines); ampholytic surfactants; semi-polar non-ionic surfactants; and mixtures thereof.

The composition can comprise optionally, a surfactancy boosting polymer consisting of amphiphilic alkoxylated grease cleaning polymers selected from a group of alkoxylated polymers having branched hydrophilic and hydrophobic properties, such as alkoxylated polyalkylenimines in the range of 0.05wt %-10wt % and/or random graft polymers typically comprising a hydrophilic backbone comprising monomers selected from the group consisting of: unsaturated C₁-C₆carboxylic acids, ethers, alcohols, aldehydes, ketones, esters, sugar units, alkoxy units, maleic anhydride, saturated polyalcohols such as glycerol, and mixtures thereof; and hydrophobic side chain(s) selected from the group consisting of: C₄-C₂₅alkyl group, polypropylene, polybutylene, vinyl ester of a saturated C₂-C₆mono-carboxylic acid, C₁-C₆alkyl ester of acrylic or methacrylic acid, and mixtures thereof.

The composition can comprise additional polymers such as soil release polymers including, for example, anionically end-capped polyesters, for example SRP1; polymers comprising at least one monomer unit selected from saccharide, dicarboxylic acid, polyol and combinations thereof, in random or block configuration; ethylene terephthalate-based polymers and co-polymers thereof in random or block configuration, for example, Repel-o-tex SF, SF-2 and SRP6, Texcare SRA100, SRA300, SRN100, SRN170, SRN240, SRN300 and SRN325, Marloquest SL; anti-redeposition polymers (0.1 wt % to 10wt %, including, for example, carboxylate polymers, such as polymers comprising at least one monomer selected from acrylic acid, maleic acid (or maleic anhydride), fumaric acid, itaconic acid, aconitic acid, mesaconic acid, citraconic acid, methylenemalonic acid, and any mixture thereof; vinylpyrrolidone homopolymer; and/or polyethylene glycol with a molecular weight in the range of from 500 to 100,000 Da); cellulosic polymer (including, for example, alkyl cellulose; alkyl alkoxyalkyl cellulose; carboxyalkyl cellulose; alkyl carboxyalkyl cellulose, examples of which include carboxymethyl cellulose, methyl cellulose, methyl hydroxyethyl cellulose, methyl carboxymethyl cellulose; and mixtures thereof); and polymeric carboxylate (such as, for example, maleate/acrylate random copolymer or polyacrylate homopolymer).

The composition can further comprise saturated or unsaturated fatty acid, preferably saturated or unsaturated C₁₂-C₂₄fatty acid (0-10 wt %); deposition aids (including, for example, polysaccharides, cellulosic polymers, polydiallyl dimethyl ammonium halides (DADMAC), and co-polymers of DADMAC with vinyl pyrrolidone, acrylamides, imidazoles, imidazolinium halides, and mixtures thereof, in random or block configuration; cationic guar gum; cationic cellulose such as cationic hydoxyethyl cellulose; cationic starch; cationic polyacylamides; and mixtures thereof.

The composition can further comprise dye transfer inhibiting agents examples of which include manganese phthalocyanine, peroxidases, polyvinylpyrrolidone polymers, polyamine N-oxide polymers, copolymers of N-vinylpyrrolidone and N-vinylimidazole, polyvinyloxazolidones and polyvinylimidazoles and/or mixtures thereof; chelating agents examples of which include ethylene-diamine-tetraacetic acid (EDTA); diethylene triamine penta methylene phosphonic acid (DTPMP); hydroxy-ethane diphosphonic acid (HEDP); ethylenediamine N,N′-disuccinic acid (EDDS); methyl glycine diacetic acid (MGDA); diethylene triamine penta acetic acid (DTPA); propylene diamine tetracetic acid (PDT A); 2-hydroxypyridine-N-oxide (HPNO); or methyl glycine diacetic acid (MGDA); glutamic acid N,N-diacetic acid (N,N-dicarboxymethyl glutamic acid tetrasodium salt (GLDA); nitrilotriacetic acid (NTA); 4,5-dihydroxy-m-benzenedisulfonic acid; citric acid and any salts thereof; N-hydroxyethylethylenediaminetri-acetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP), and derivatives thereof.

The composition can further comprise silicone or fatty-acid based suds suppressors; an enzyme stabilizer; hueing dyes, calcium and magnesium cations, visual signaling ingredients, anti-foam (0.001 to about 4.0 wt %), and/or structurant/thickener (0.01-5 wt %) selected from the group consisting of diglycerides, triglycerides, ethylene glycol distearate, microcrystalline cellulose, cellulose based materials, microfiber cellulose, biopolymers, xanthan gum, gellan gum, and mixtures thereof.

In some embodiments, the composition is a high density powder (HDD) composition comprising at least one polypeptide described herein. The HDD powder laundry detergent can comprise a detersive surfactant including anionic detersive surfactants (selected from linear or branched or random chain, substituted or unsubstituted alkyl sulphates, alkyl sulphonates, alkyl alkoxylated sulphate, alkyl phosphates, alkyl phosphonates, alkyl carboxylates and/or mixtures thereof), non-ionic detersive surfactant (selected from 1 linear or branched or random chain, substituted or unsubstituted C₈-C₁₈ alkyl ethoxylates, and/or C₆-C₁₂ alkyl phenol alkoxylates), cationic detersive surfactants (selected from alkyl pyridinium compounds, alkyl quaternary ammonium compounds, alkyl quaternary phosphonium compounds, alkyl ternary sulphonium compounds, and mixtures thereof); zwitterionic and/or amphoteric detersive surfactants (selected from alkanolamine sulpho-betaines); ampholytic surfactants; semi-polar non-ionic surfactants and mixtures thereof; builders (phosphate free builders, e,g., zeolite builders examples of which include zeolite A, zeolite X, zeolite P and zeolite MAP in the range of 0 to less than 10 wt %); phosphate builders, e.g., sodium tri-polyphosphate in the range of 0 to less than 10 wt %; citric acid, citrate salts and nitrilotriacetic acid or salt thereof in the range of less than 15 wt %; silicate salt (sodium or potassium silicate or sodium meta-silicate in the range of 0 to less than 10 wt % or layered silicate (SKS-6)); carbonate salt (sodium carbonate and/or sodium bicarbonate in the range of 0 to less than 10 wt %); and bleaching agents (photobleaches, e.g., sulfonated zinc phthalocyanines, sulfonated aluminum phthalocyanines, xanthenes dyes, and mixtures thereof); hydrophobic or hydrophilic bleach activators (e.g., dodecanoyl oxybenzene sulfonate, decanoyl oxybenzene sulfonate, decanoyl oxybenzoic acid or salts thereof, 3,5,5-trimethy hexanoyl oxybenzene sulfonate, tetraacetyl ethylene diamine-TAED, and nonanoyloxybenzene sulfonate-NOBS, nitrile quats, and mixtures thereof); hydrogen peroxide; sources of hydrogen peroxide (inorganic perhydrate salts, e.g., mono or tetra hydrate sodium salt of perborate, percarbonate, persulfate, perphosphate, or persilicate); preformed hydrophilic and/or hydrophobic peracids (selected from percarboxylic acids and salts, percarbonic acids and salts, perimidic acids and salts, peroxymonosulfuric acids and salts, and mixtures thereof); and/or bleach catalyst (e.g., imine bleach boosters, such as iminium cations and polyions; iminium zwitterions; modified amines; modified amine oxides; N-sulphonyl imines; N-phosphonyl imines; N-acyl imines; thiadiazole dioxides; perfluoroimines; cyclic sugar ketones and mixtures thereof), metal-containing bleach catalyst (e.g., copper, iron, titanium, ruthenium, tungsten, molybdenum, or manganese cations along with an auxiliary metal cations such as zinc or aluminum and a sequestrate such as ethylenediaminetetraacetic acid, ethylenediaminetetra(methylenephosphonic acid) and water-soluble salts thereof).

The composition can further comprise additional detergent ingredients including perfume microcapsules, starch encapsulated perfume accord, an enzyme stabilizer, hueing agents, additional polymers including fabric integrity and cationic polymers, dye lock ingredients, fabric-softening agents, brighteners (for example C.I. Fluorescent brighteners), flocculating agents, chelating agents, alkoxylated polyamines, fabric deposition aids, and/or cyclodextrin.

In some embodiments, the composition is an ADW detergent composition comprising at least one polypeptide described herein. The ADW detergent composition can comprise two or more non-ionic surfactants selected from ethoxylated non-ionic surfactants, alcohol alkoxylated surfactants, epoxy-capped poly(oxyalkylated) alcohols, and amine oxide surfactants present in amounts from 0-10% by wt; builders in the range of 5-60% by wt. comprising either phosphate (mono-phosphates, di-phosphates, tri-polyphosphates or oligomeric-poylphosphates), sodium tripolyphosphate-STPP or phosphate-free builders (amino acid based compounds, e.g., MGDA (methyl-glycine-diacetic acid) and salts and derivatives thereof, GLDA (glutamic-N,Ndiacetic acid) and salts and derivatives thereof, IDS (iminodisuccinic acid) and salts and derivatives thereof, carboxy methyl inulin and salts and derivatives thereof and mixtures thereof, nitrilotriacetic acid (NTA), diethylene triamine penta acetic acid (DTPA), and B-alaninediacetic acid (B-ADA) and their salts), homopolymers and copolymers of poly-carboxylic acids and their partially or completely neutralized salts, monomeric polycarboxylic acids and hydroxycarboxylic acids and their salts in the range of 0.5-50% by wt; sulfonated/carboxylated polymers (provide dimensional stability to the product) in the range of about 0.1 to about 50% by wt; drying aids in the range of about 0.1 to about 10% by wt (selected from polyesters, especially anionic polyesters optionally together with further monomers with 3-6 functionalities which are conducive to polycondensation, specifically acid, alcohol or ester functionalities, polycarbonate-, polyurethane- and/or polyurea-polyorganosiloxane compounds or precursor compounds thereof of the reactive cyclic carbonate and urea type); silicates in the range from about 1 to about 20% by wt (sodium or potassium silicates, e.g., sodium disilicate, sodium meta-silicate and crystalline phyllosilicates); bleach-inorganic (e.g., perhydrate salts such as perborate, percarbonate, perphosphate, persulfate and persilicate salts) and organic (e.g., organic peroxyacids including diacyl and tetraacylperoxides, especially diperoxydodecanedioc acid, diperoxytetradecanedioc acid, and diperoxyhexadecanedioc acid); bleach activator-organic peracid precursors in the range from about 0.1 to about 10% by wt; bleach catalysts (selected from manganese triazacyclononane and related complexes, Co, Cu, Mn and Fe bispyridylamine and related complexes, and pentamine acetate cobalt(III) and related complexes); metal care agents in the range from about 0.1-5% by wt (selected from benzatriazoles, metal salts and complexes, and silicates); enzymes in the range from about 0.01-5.0 mg of active enzyme per gram of ADW detergent composition (acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, galactanases, glucoamylases, hemicellulases, hexosaminidases, hyaluronidases, keratinases, laccases, lactases, ligninases, lipases, lipoxygenases, mannanases, oxidases, pectate lyases, pectin acetyl esterases, pectinases, pentosanases, peroxidases, phenoloxidases, phosphatases, phospholipases, phytases, polygalacturonases, proteases, pullulanases, reductases, rhamnogalacturonases, beta-glucanases, tannases, transglutaminases, xylan acetyl-esterases, xylanases, xyloglucanases, xylosidases, and mixtures thereof); and enzyme stabilizer components.

Exemplary ADW compositions are provided in the Examples below, or in the following Table.

### Exemplary ADW Composition

| Ingredient | Weight in grams |
|---|---|
| Bleach Activator | 0.22 |
| (tetraacetylethylenediamine; TAED) | |
| SKS-6 sodium disilicate | 0.8 |
| (Na₂Si₂O₅) | |
| HEDP | 0.93 |
| Sodium carbonate | 1.5 |
| MGDA | 7.01 |
| Sulfonic acid group-containing polymer | 0.80 |
| (Acusol ™ 588) | |
| Sodium percarbonate | 3.50 |
| Bleach catalyst (Manganese 1,4,7- | 0.256 |
| triazacyclononane; MnTACN) | |
| LUTENSOL ® TO7 | 0.90 |
| PLURAFAC ® SLF 180 | 0.75 |
| Dipropylene glycol | 0.40 |
| Minors | balance |
| Total % of full dose | 100 |

More embodiments are directed to compositions and methods of treating fabrics (e.g., to desize a textile) using at least one polypeptide or composition comprising at least one polypeptide described herein. Fabric-treating methods are well known in the art (see, e.g., U.S. Pat. No. 6,077,316). For example, the feel and appearance of a fabric can be improved by a method comprising contacting the fabric with a polypeptide described herein in a solution. The fabric can be treated with the solution under pressure.

At least one polypeptide described herein can be applied during or after weaving a textile, during the desizing stage, or one or more additional fabric processing steps. During the weaving of textiles, the threads are exposed to considerable mechanical strain. Prior to weaving on mechanical looms, warp yarns are often coated with sizing starch or starch derivatives to increase their tensile strength and to prevent breaking. At least one polypeptide described herein can be applied during or after weaving to remove the sizing starch or starch derivatives. After weaving, the polypeptide can be used to remove the size coating before further processing the fabric to ensure a homogeneous and wash-proof result. At least one polypeptide described herein can be used alone or with other desizing chemical reagents and/or desizing enzymes to desize fabrics, including cotton-containing fabrics, as detergent additives, e.g., in aqueous compositions. An amylase also can be used in compositions and methods for producing a stonewashed look on indigo-dyed denim fabric and garments. For the manufacture of clothes, the fabric can be cut and sewn into clothes or garments, which are afterwards finished. In particular, for the manufacture of denim jeans, different enzymatic finishing methods have been developed. The finishing of denim garment normally is initiated with an enzymatic desizing step, during which garments are subjected to the action of proteolytic enzymes to provide softness to the fabric and make the cotton more accessible to the subsequent enzymatic finishing steps. At least one polypeptide described herein can be used in methods of finishing denim garments (e.g., a “bio-stoning process”), enzymatic desizing and providing softness to fabrics, and/or finishing process. In another embodiment, the polypeptides provided herein having serine protease activity, where the polypeptide comprises an amino acid sequence with at least 75% identity with the amino acid sequence selected from the group consisting of SEQ ID NOs: 2, 6, 7, 8, and 13 can further be used in feed additive compositions and methods related thereto. A feed additive composition for use in animal feed may comprise a serine protease having at least 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NOs: 2, 6, 7, 8, and 13 used either alone or in combination with at least one direct fed microbial (such as a Bacillus direct fed microbial, for example, Bacillus subtilis or Bacillus amyloliquefaciens), and at least one component selected from the group consisting of a protein, a peptide, sucrose, lactose, sorbitol, glycerol, propylene glycol, sodium chloride, sodium sulfate, sodium acetate, sodium citrate, sodium formate, sodium sorbate, potassium chloride, potassium sulfate, potassium acetate, potassium citrate, potassium formate, potassium acetate, potassium sorbate, magnesium chloride, magnesium sulfate, magnesium acetate, magnesium citrate, magnesium formate, magnesium sorbate, sodium metabi sulfite, methyl paraben and propyl paraben. In still another embodiment, there is disclosed a granulated feed additive composition for use in animal feed comprising a serine protease having at least 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NOs: 2, 6, 7, 8, and 13, used either alone or in combination with at least one direct fed microbial (such as a Bacillus direct fed microbial, for example, Bacillus subtilis or Bacillus amyloliquefaciens), wherein the granulated feed additive composition comprises particles produced by a process selected from the group consisting of high shear granulation, drum granulation, extrusion, spheronization, fluidized bed agglomeration, fluidized bed spray coating, spray drying, freeze drying, prilling, spray chilling, spinning disk atomization, coacervation, tableting, or any combination of the above processes. Furthermore, the particles of the granulated feed additive composition can have a mean diameter of greater than 50 microns and less than 2000 microns.

The feed additive composition can be a liquid form and the liquid form can also be said suitable for spray-drying on a feed pellet.

“Animal feeds” may include plant material such as corn, wheat, sorghum, soybean, canola, sunflower or mixtures of any of these plant materials or plant protein sources for poultry, pigs, ruminants, aquaculture and pets. It is contemplated that animal performance parameters, such as growth, feed intake and feed efficiency, but also improved uniformity, reduced ammonia concentration in the animal house and consequently improved welfare and health status of the animals will be improved. More specifically, as used herein, “animal performance” may be determined by the feed efficiency and/or weight gain of the animal and/or by the feed conversion ratio and/or by the digestibility of a nutrient in a feed (e.g. amino acid digestibility) and/or digestible energy or metabolizable energy in a feed and/or by nitrogen retention and/or by the animal's ability to avoid the negative effects of necrotic enteritis and/or by the immune response of the subject. Preferably “animal performance” is determined by feed efficiency and/or weight gain of the animal and/or by the feed conversion ratio. By “improved animal performance” it is meant that there is increased feed efficiency, and/or increased weight gain and/or reduced feed conversion ratio and/or improved digestibility of nutrients or energy in a feed and/or by improved nitrogen retention and/or by improved ability to avoid the negative effects of necrotic enteritis and/or by an improved immune response in the subject resulting from the use of feed additive composition of the present invention in feed in comparison to feed which does not comprise said feed additive composition.

Preferably, by “improved animal performance” it is meant that there is increased feed efficiency and/or increased weight gain and/or reduced feed conversion ratio. As used herein, the term “feed efficiency” refers to the amount of weight gain in an animal that occurs when the animal is fed ad-libitum or a specified amount of food during a period of time.

By “increased feed efficiency” it is meant that the use of a feed additive composition according the present invention in feed results in an increased weight gain per unit of feed intake compared with an animal fed without said feed additive composition being present. As used herein, the term “feed conversion ratio” refers to the amount of feed fed to an animal to increase the weight of the animal by a specified amount. An improved feed conversion ratio means a lower feed conversion ratio. By “lower feed conversion ratio” or “improved feed conversion ratio” it is meant that the use of a feed additive composition in feed results in a lower amount of feed being required to be fed to an animal to increase the weight of the animal by a specified amount compared to the amount of feed required to increase the weight of the animal by the same amount when the feed does not comprise said feed additive composition.

“Nutrient digestibility” as used herein means the fraction of a nutrient that disappears from the gastro-intestinal tract or a specified segment of the gastro-intestinal tract, e.g. the small intestine. Nutrient digestibility may be measured as the difference between what is administered to the subject and what comes out in the feces of the subject, or between what is administered to the subject and what remains in the digesta on a specified segment of the gastro intestinal tract, e.g. the ileum. Nutrient digestibility as used herein may be measured by the difference between the intake of a nutrient and the excreted nutrient by means of the total collection of excreta during a period of time; or with the use of an inert marker that is not absorbed by the animal, and allows the researcher calculating the amount of nutrient that disappeared in the entire gastro-intestinal tract or a segment of the gastro-intestinal tract. Such an inert marker may be titanium dioxide, chromic oxide or acid insoluble ash. Digestibility may be expressed as a percentage of the nutrient in the feed, or as mass units of digestible nutrient per mass units of nutrient in the feed. Nutrient digestibility as used herein encompasses starch digestibility, fat digestibility, protein digestibility, and amino acid digestibility.

“Energy digestibility” as used herein means the gross energy of the feed consumed minus the gross energy of the feces or the gross energy of the feed consumed minus the gross energy of the remaining digesta on a specified segment of the gastro-intestinal tract of the animal, e.g. the ileum. Metabolizable energy as used herein refers to apparent metabolizable energy and means the gross energy of the feed consumed minus the gross energy contained in the feces, urine, and gaseous products of digestion. Energy digestibility and metabolizable energy may be measured as the difference between the intake of gross energy and the gross energy excreted in the feces or the digesta present in specified segment of the gastro-intestinal tract using the same methods to measure the digestibility of nutrients, with appropriate corrections for nitrogen excretion to calculate metabolizable energy of feed.

In some embodiments, the compositions described herein can improve the digestibility or utilization of dietary hemicellulose or fiber in a subject. In some embodiments, the subject is a pig.

The term “carcass yield” as used herein means the amount of carcass as a proportion of the live body weight, after a commercial or experimental process of slaughter. The term carcass means the body of an animal that has been slaughtered for food, with the head, entrails, part of the limbs, and feathers or skin removed. The term meat yield as used herein means the amount of edible meat as a proportion of the live body weight, or the amount of a specified meat cut as a proportion of the live body weight.

An “increased weight gain” refers to an animal having increased body weight on being fed feed comprising a feed additive composition compared with an animal being fed a feed without said feed additive composition being present.

The term “animal” as used herein includes all non-ruminant and ruminant animals. In a particular embodiment, the animal is a non-ruminant animal, such as a horse and a mono-gastric animal. Examples of mono-gastric animals include, but are not limited to, pigs and swine, such as piglets, growing pigs, sows; poultry such as turkeys, ducks, chicken, broiler chicks, layers; fish such as salmon, trout, tilapia, catfish and carps; and crustaceans such as shrimps and prawns. In a further embodiment the animal is a ruminant animal including, but not limited to, cattle, young calves, goats, sheep, giraffes, bison, moose, elk, yaks, water buffalo, deer, camels, alpacas, llamas, antelope, pronghorn and nilgai.

The terms “animal feed composition,” “feed”, “feedstuff and “fodder” are used interchangeably and can comprise one or more feed materials selected from the group comprising a) cereals, such as small grains (e.g., wheat, barley, rye, oats and combinations thereof) and/or large grains such as maize or sorghum; b) by products from cereals, such as corn gluten meal, Distillers Dried Grains with Solubles (DDGS) (particularly corn based Distillers Dried Grains with Solubles (cDDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; and/or e) minerals and vitamins.

The serine proteases described herein or a feed additive composition may be used as, or in the preparation of, a feed. The terms “feed additive composition” and “enzyme composition” are used interchangeably herein. The feed may be in the form of a solution or as a solid or as a semi-solid depending on the use and/or the mode of application and/or the mode of administration.

When used as, or in the preparation of, a feed, such as functional feed, the enzyme or feed additive composition described herein may be used in conjunction with one or more of: a nutritionally acceptable carrier, a nutritionally acceptable diluent, a nutritionally acceptable excipient, a nutritionally acceptable adjuvant, a nutritionally active ingredient. For example, there be mentioned at least one component selected from the group consisting of a protein, a peptide, sucrose, lactose, sorbitol, glycerol, propylene glycol, sodium chloride, sodium sulfate, sodium acetate, sodium citrate, sodium formate, sodium sorbate, potassium chloride, potassium sulfate, potassium acetate, potassium citrate, potassium formate, potassium acetate, potassium sorbate, magnesium chloride, magnesium sulfate, magnesium acetate, magnesium citrate, magnesium formate, magnesium sorbate, sodium metabi sulfite, methyl paraben and propyl paraben.

In a preferred embodiment the enzyme or feed additive composition of the present invention is admixed with a feed component to form a feedstuff. The term “feed component” as used herein means all or part of the feedstuff. Part of the feedstuff may mean one constituent of the feedstuff or more than one constituent of the feedstuff, e.g. 2 or 3 or 4 or more. In one embodiment the term “feed component” encompasses a premix or premix constituents.

Preferably, the feed may be a fodder, or a premix thereof, a compound feed, or a premix thereof. A feed additive composition may be admixed with a compound feed, a compound feed component or to a premix of a compound feed or to a fodder, a fodder component, or a premix of a fodder. Any feedstuff described herein may comprise one or more feed materials selected from the group comprising a) cereals, such as small grains (e.g., wheat, barley, rye, oats, triticale and combinations thereof) and/or large grains such as maize or sorghum; b) by products from cereals, such as corn gluten meal, wet-cake (particularly corn based wet-cake), Distillers Dried Grains (DDG) (particularly corn based Distillers Dried Grains (cDDG)), Distillers Dried Grains with Solubles (DDGS) (particularly corn based Distillers Dried Grains with Solubles (cDDGS)), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; e) minerals and vitamins.

A feedstuff described herein may contain at least 30%, at least 40%, at least 50% or at least 60% by weight corn and soybean meal or corn and full fat soy, or wheat meal or sunflower meal. For example, a feedstuff may contain between about 5 to about 40% corn DDGS. For poultry, the feedstuff on average may contain between about 7 to 15% corn DDGS. For swine (pigs), the feedstuff may contain on average 5 to 40% corn DDGS. It may also contain corn as a single grain, in which case the feedstuff may comprise between about 35% to about 80% corn.

The term “fodder” as used herein means any food which is provided to an animal (rather than the animal having to forage for it themselves). Fodder encompasses plants that have been cut. Furthermore, fodder includes silage, compressed and pelleted feeds, oils and mixed rations, and also sprouted grains and legumes. Fodder may be obtained from one or more of the plants selected from: corn (maize), alfalfa (Lucerne), barley, birdsfoot trefoil, brassicas, Chau moellier, kale, rapeseed (canola), rutabaga (swede), turnip, clover, alsike clover, red clover, subterranean clover, white clover, fescue, brome, millet, oats, sorghum, soybeans, trees (pollard tree shoots for tree-hay), wheat, and legumes.

The term “compound feed” means a commercial feed in the form of a meal, a pellet, nuts, cake or a crumble. Compound feeds may be blended from various raw materials and additives. These blends are formulated according to the specific requirements of the target animal. Compound feeds can be complete feeds that provide all the daily required nutrients, concentrates that provide a part of the ration (protein, energy) or supplements that only provide additional micronutrients, such as minerals and vitamins. The main ingredients used in compound feed are the feed grains, which include corn, wheat, canola meal, rapeseed meal, lupin, soybeans, sorghum, oats, and barley.

Suitably a “premix” as referred to herein may be a composition composed of microingredients such as vitamins, minerals, chemical preservatives, antibiotics, fermentation products, and other essential ingredients. Premixes are usually compositions suitable for blending into commercial rations.

As used herein the term “contacted” refers to the indirect or direct application of a serine protease enzyme (or composition comprising the serine protease) to a product (e.g. the feed). Examples of application methods which may be used, include, but are not limited to, treating the product in a material comprising the feed additive composition, direct application by mixing the feed additive composition with the product, spraying the feed additive composition onto the product surface or dipping the product into a preparation of the feed additive composition. In one embodiment the feed additive composition of the present invention is preferably admixed with the product (e.g. feedstuff).

Alternatively, the feed additive composition may be included in the emulsion or raw ingredients of a feedstuff. For some applications, it is important that the composition is made available on or to the surface of a product to be affected/treated. This allows the composition to impart a performance benefit. In some aspects, the serine proteases described are used for the pre- treatment of food or feed. For example, the feed having 10-300% moisture is mixed and incubated with the proteases at 5-80° C., preferably at 25-50° C., more preferably between 30-45° C. for 1 min to 72 hours under aerobic conditions or 1 day to 2 months under anaerobic conditions. The pre-treated material can be fed directly to the animals (so called liquid feeding). The pre-treated material can also be steam pelleted at elevated temperatures of 60-120° C. The proteases can be impregnated to feed or food material by a vacuum coater. Serine proteases (or composition comprising the serine proteases) may be applied to intersperse, coat and/or impregnate a product (e.g. feedstuff or raw ingredients of a feedstuff) with a controlled amount of said enzyme.

Preferably, the feed additive composition will be thermally stable to heat treatment up to about 70° C.; up to about 85° C.; or up to about 95° C. The heat treatment may be performed for up to about 1 minute; up to about 5 minutes; up to about 10 minutes; up to about 30 minutes; up to about 60 minutes. The term thermally stable means that at least about 75% of the enzyme components and/or DFM that were present/active in the additive before heating to the specified temperature are still present/active after it cools to room temperature. Preferably, at least about 80%) of the protease component and/or DFM comprising one or more bacterial strains that were present and active in the additive before heating to the specified temperature are still present and active after it cools to room temperature. In a particularly preferred embodiment the feed additive composition is homogenized to produce a powder.

Alternatively, the feed additive composition is formulated to granules as described in WO2007/044968 (referred to as TPT granules) incorporated herein by reference.

In another preferred embodiment when the feed additive composition is formulated into granules the granules comprise a hydrated barrier salt coated over the protein core. The advantage of such salt coating is improved thermo-tolerance, improved storage stability and protection against other feed additives otherwise having adverse effect on the at least one protease and/or DFM comprising one or more bacterial strains. Preferably, the salt used for the salt coating has a water activity greater than 0.25 or constant humidity greater than 60%>at 20° C.

The method of preparing a feed additive composition may also comprise the further step of pelleting the powder. The powder may be mixed with other components known in the art. The powder, or mixture comprising the powder, may be forced through a die and the resulting strands are cut into suitable pellets of variable length.

A method of preparing serine proteases (or composition comprising the serine proteases) may also comprise the further step of pelleting the powder. The powder may be mixed with other components known in the art. The powder, or mixture comprising the powder, may be forced through a die and the resulting strands are cut into suitable pellets of variable length.

Optionally, the pelleting step may include a steam treatment, or conditioning stage, prior to formation of the pellets. The mixture comprising the powder may be placed in a conditioner, e.g. a mixer with steam injection. The mixture is heated in the conditioner up to a specified temperature, such as from 60-100° C., typical temperatures would be 70° C., 80° C., 85° C., 90° C. or 95° C. The residence time can be variable from seconds to minutes and even hours. Such as 5 seconds, 10 seconds, 15 seconds, 30 seconds, 1 minutes 2 minutes., 5 minutes, 10 minutes, 15 minutes, 30 minutes and 1 hour. It will be understood that the serine proteases (or composition comprising the serine proteases) described herein are suitable for addition to any appropriate feed material. It will be understood by the skilled person that different animals require different feedstuffs, and even the same animal may require different feedstuffs, depending upon the purpose for which the animal is reared.

Optionally, the feedstuff may also contain additional minerals such as, for example, calcium and/or additional vitamins. In some embodiments, the feedstuff is a corn soybean meal mix.

Feedstuff is typically produced in feed mills in which raw materials are first ground to a suitable particle size and then mixed with appropriate additives. The feedstuff may then be produced as a mash or pellets; the later typically involves a method by which the temperature is raised to a target level and then the feed is passed through a die to produce pellets of a particular size. The pellets are allowed to cool. Subsequently liquid additives such as fat and enzyme may be added. Production of feedstuff may also involve an additional step that includes extrusion or expansion prior to pelleting, in particular by suitable techniques that may include at least the use of steam. The feed additive composition and/or the feedstuff comprising same may be used in any suitable form. The feed additive composition may be used in the form of solid or liquid preparations or alternatives thereof. Examples of solid preparations include powders, pastes, boluses, capsules, pellets, tablets, dusts, and granules which may be wettable, spray-dried or freeze-dried. Examples of liquid preparations include, but are not limited to, aqueous, organic or aqueous-organic solutions, suspensions and emulsions. In some applications, the feed additive compositions may be mixed with feed or administered in the drinking water.

A feed additive composition, comprising admixing a protease as taught herein with a feed acceptable carrier, diluent or excipient, and (optionally) packaging.

In some embodiments, serine protease can be present in the feedstuff in the range of 1 ppb (parts per billion) to 10% (w/w) based on pure enzyme protein. In some embodiments, the protease is present in the feedstuff is in the range of 1-100 ppm (parts per million). A preferred dose can be 1-20 g of serine protease per ton of feed product or feed composition or a final dose of 1-20 ppm serine protease in final product.

Preferably, the serine protease is present in the feedstuff should be at least about 200 PU/kg or at least about 300 PU/kg feed or at least about 400 PU/kg feed or at least about 500 PU/kg feed or at least about 600 PU/kg feed, at least about 700 PU/kg feed, at least about 800 PU/kg feed, at least about 900 PU/kg feed or at least about 1000 PU/kg feed, or at least about 1500 PU/kg feed, or at least about 2000 PU/kg feed or at least about 2500 PU/kg feed, or at least about 3000 PU/kg feed, or at least about 3500 PU/kg feed, or at least about 4000 PU/kg feed, or at least about 4500 PU/kg feed, or at least about 5000 PU/kg feed.

In another aspect, serine protease can be present in the feedstuff at less than about 60,000 PU/kg feed, or at less than about 70,000 PU/kg feed, or at less than about 80,000 PU/kg feed, or at less than about 90,000 PU/kg feed, or at less than about 100,000 PU/kg feed, or at less than about 200,000 PU/kg feed, or at less than about 60000 PU/kg feed, or at less than about 70000 PU/kg feed. Ranges can include, but are not limited to, any combination of the lower and upper ranges discussed above.

It will be understood that one protease unit (PU) is the amount of enzyme that liberates 2.3 micrograms of phenolic compound (expressed as tyrosine equivalents) from a casein substrate per minute at pH 10.0 at 50° C. This may be referred to as the assay for determining 1 PU. Formulations comprising any of serine protease and compositions described herein may be made in any suitable way to ensure that the formulation comprises active enzymes. Such formulations may be as a liquid, a dry powder or a granule. Preferably, the feed additive composition is in a liquid form suitable for spray-drying on a feed pellet.

Dry powder or granules may be prepared by means known to those skilled in the art, such as, high shear granulation, drum granulation, extrusion, spheronization, fluidized bed agglomeration, or fluidized bed spray.

The serine proteases and compositions described herein may be coated, for example encapsulated. In one embodiment, the coating protects the enzymes from heat and may be considered a thermo-protectant.

Feed additive composition described herein can be formulated to a dry powder or granules as described in WO2007/044968 (referred to as TPT granules) or WO 1997/016076 or WO1992/012645 (each of which is incorporated herein by reference).

In one embodiment the feed additive composition may be formulated to a granule for feed compositions comprising: a core; an active agent; and at least one coating, the active agent of the granule retaining at least 50% activity, at least 60%>activity, at least 70% activity, at least 80%) activity after conditions selected from one or more of a) a feed pelleting process, b) a steam-heated feed pretreatment process, c) storage, d) storage as an ingredient in an unpelleted mixture, and e) storage as an ingredient in a feed base mix or a feed premix comprising at least one compound selected from trace minerals, organic acids, reducing sugars, vitamins, choline chloride, and compounds which result in an acidic or a basic feed base mix or feed premix.

With regard to the granule at least one coating may comprise a moisture hydrating material that constitutes at least 55% w/w of the granule; and/or at least one coating may comprise two coatings. The two coatings may be a moisture hydrating coating and a moisture barrier coating. In some embodiments, the moisture hydrating coating may be between 25% and 60%) w/w of the granule and the moisture barrier coating may be between 2% and 15% w/w of the granule. The moisture hydrating coating may be selected from inorganic salts, sucrose, starch, and maltodextrin and the moisture barrier coating may be selected from polymers, gums, whey and starch. The granule may be produced using a feed pelleting process and the feed pretreatment process may be conducted between 70° C. and 95° C. for up to several minutes, such as between 85° C. and 95° C.

The feed additive composition may be formulated to a granule for animal feed comprising: a core; an active agent, the active agent of the granule retaining at least 80% activity after storage and after a steam-heated pelleting process where the granule is an ingredient; a moisture barrier coating; and a moisture hydrating coating that is at least 25% w/w of the granule, the granule having a water activity of less than 0.5 prior to the steam-heated pelleting process.

The granule may have a moisture barrier coating selected from polymers and gums and the moisture hydrating material may be an inorganic salt. The moisture hydrating coating may be between 25% and 45% w/w of the granule and the moisture barrier coating may be between 2% and 10% w/w of the granule. The granule may be produced using a steam-heated pelleting process which may be conducted between 85° C. and 95° C. for up to several minutes.

Alternatively, the composition is in a liquid formulation suitable for consumption preferably such liquid consumption contains one or more of the following: a buffer, salt, sorbitol and/or glycerol.

Also, the feed additive composition may be formulated by applying, e.g. spraying, the enzyme(s) onto a carrier substrate, such as ground wheat for example. In one embodiment the feed additive composition may be formulated as a premix. By way of example only the premix may comprise one or more feed components, such as one or more minerals and/or one or more vitamins. In one embodiment a direct fed microbial (“DFM”) and/or serine proteases are formulated with at least one physiologically acceptable carrier selected from at least one of maltodextrin, limestone (calcium carbonate), cyclodextrin, wheat or a wheat component, sucrose, starch, Na₂SO₄, Talc, PVA, sorbitol, benzoate, sorbate, glycerol, sucrose, propylene glycol, 1,3-propane diol, glucose, parabens, sodium chloride, citrate, acetate, phosphate, calcium, metabisulfite, formate and mixtures thereof.

Non-limiting examples of compositions and methods disclosed herein include the following embodiments:

### EXAMPLES

### Example 1

### Cloning of Isoptericola sp. Protease IspPro1

Isoptericola sp. was selected as a potential source of enzymes which may be useful in various industrial applications. Genomic DNA was obtained by first growing the strain on Heart Infusion agar plate (Difco) at 37° C. for 24 hr. Cell material was scraped from the plate and used to prepare genomic DNA with the ZF Fungal/Bacterial DNA miniprep kit from Zymo (Cat No. D6005). The genomic DNA was then used for genome sequencing. The genome of the Isoptericola sp. was sequenced by BaseClear (Leiden, The Netherlands) using the Illumina's next generation sequencing technology. After assembly of the data, contigs were annotated by BioXpr (Namur, Belgium). The gene for a trypsin-like serine protease (IspPro1) was identified and its nucleotide and protein sequences are provided in SEQ ID NO: 1 and SEQ ID NO: 2, respectively. IspPro1 has an N-terminal signal peptide as predicted by SignalP version 4.0 (Nordahl Petersen et al. (2011) Nature Methods, 8:785-786), suggesting that it is a secreted enzyme.

### Example 2

### Expression of IspPro1 Protease

The DNA sequence of the propeptide-mature form of IspPro1 was synthesized and inserted into the Bacillus subtilis expression vector p2JM103BBI (Vogtentanz, Protein Expr Purif, 55:40-52, 2007) by Generay (Shanghai, China), resulting in plasmid pGX413(AprE-IspPro1). Ligation of the gene encoding the trypsin-like serine protease IspPro1 into the digested vector resulted in the addition of three codons (Ala-Gly-Lys) between the 3′ end of the B. subtilis AprE signal sequence and the 5′ end of the propeptide-mature sequence. The synthetic gene has an alternative start codon (GTG). The resulting expression vector contains an AprE promoter (SEQ ID NO: 3), an AprE signal sequence (SEQ ID NO: 4) used to direct target protein secretion in B. subtilis, and the synthetic nucleotide sequence encoding the predicted propeptide and mature regions of IspPro1. The nucleotide sequence of the synthetic AprE-IspPro1 gene is provided in SEQ ID NO: 5 and the related translation product is shown in SEQ ID NO: 6. Expression in Bacillus hosts is expected to result in processing of the IspPro1 precursor protein sequence.

The expression plasmid was transformed into a suitable B. subtilis host and the transformed cells were spread on Luria Agar plates supplemented with 5 ppm Chloramphenicol and 1.2% skim milk (Cat #232100, Difco). Colonies with the largest clear halos on the plates were selected and subjected to fermentation in a 250 mL shake flask with MBD medium (a MOPS based defined medium).

To purify IspPro1 protease, the crude material from shake flasks was concentrated and ammonium sulfate added to the final concentration of 1 M. The solution was loaded onto a HiPrep™ Phenyl FF (Cytiva, Marlborough, MA, USA) column pre-equilibrated with 20 mM sodium phosphate (pH7.0) supplemented with additional 1 M ammonium sulfate (Buffer A). The target protein was eluted from the column with 0.75 M ammonium sulfate. The corresponding proteolytically active fractions were pooled, concentrated and buffer exchanged into 20 mM Tris (pH 8.0) supplemented with 10% propylene glycol (Buffer B), using a VivaFlow 200 ultra-filtration device (Sartorius Stedim). The resulting solution was applied to a HiPrep™ Q FF 16/10 column (Cytiva, Marlborough, MA, USA) pre-equilibrated with Buffer B. The target protein flowed through from the column. The resulting proteolytically active fractions were then pooled and concentrated via the 10K Amicon Ultra devices and stored in 40% glycerol at −20° C. until usage.

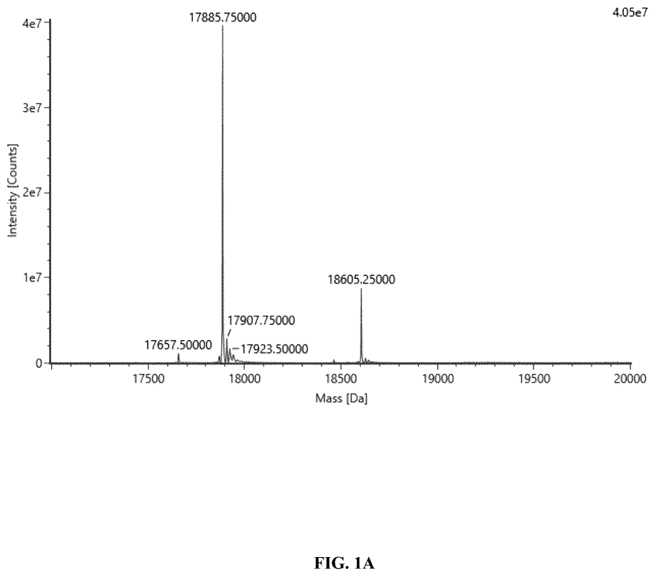

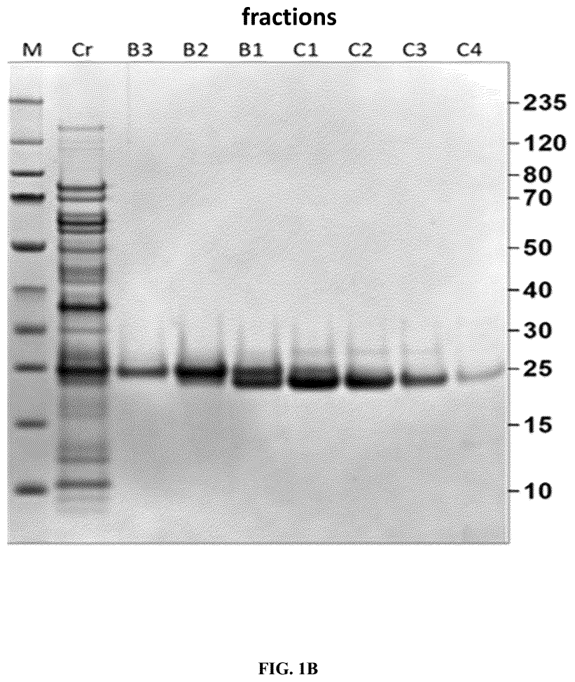

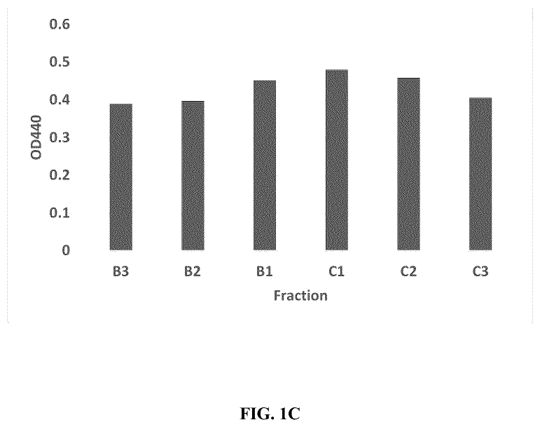

SDS-PAGE analysis of purified IspPro1 protease samples revealed the presence of two major bands on the gel that ran close together at the expected apparent mass of the processed molecule. The presence of the two molecular species was confirmed by mass spectrometry analysis, which detected molecules with masses of 17,886 Da and 18,605 Da (FIG. 1A). Athirty milligram of quantify of the purified sample was analyzed by gel filtration using a Superdex 75 column (Hi Load™ 26/60 Superdex 75, Cytiva, Marlborough, MA, USA). The sample was eluted with 20 mM sodium phosphate, pH 7.0 containing 150 mM NaCl and 10% propylene glycol and fractions were collected. Proteolytically active fractions contained pure protein corresponding to either the larger (18,605 Da) or the smaller (17,886 Da) molecular species or contained a mixture of both (fraction B1), as judged by SDS-PAGE analysis shown on FIG. 1B. The specific activity of each column fraction shown in FIG. 1B was tested at 5 ppm final enzyme concentration in 50 mM HEPES (pH 8), using azo-casein (Cat #74H7165, Megazyme, Bray, Ireland) as the substrate. Prior to the reaction, each fraction was diluted with water to produce 100 ppm stock. The azo-casein was dissolved in 50 mM HEPES (pH 8) to a final concentration of 0.75% (w/v). To initiate the reaction, 5 μL of the diluted enzyme (or water alone as the blank control) was added to a non-binding 96-well Microtiter Plate (96-MTP) (Corning Life Sciences, #3641) placed on ice, which was followed by the addition of 95 μL of the substrate solution. After sealing the 96-MTP, the reaction was carried out in a Thermomixer (Eppendorf, Sigma-Aldrich, St Louis, MO, USA) at 45° C. and 650 rpm for 15 min. The reaction was terminated by adding 100 μL of 8% Trichloroacetic Acid (TCA). Following equilibration (5 min at the room temperature) and subsequent centrifugation (2000 g for 10 min at 4° C.), 100 μL supernatant was transferred to a new 96-MTP, and its absorbance at 440 nm (A₄₄₀) was measured using a SpectraMax 190 (Molecular Devices, San Jose, CA, USA). Net A₄₄₀ was calculated by subtracting A₄₄₀ of the blank control from that of enzyme. Each value was the mean of duplicate assays, and the value varied no more than 5%. As shown in FIG. 1C, the fractions from the gel filtration chromatography had comparable specific activities.

The amino acid sequences of the two protein bands (shown on FIG. 1A and 1B) in the purified IspPro1 protease sample, determined by tandem mass spectrometry, are listed as SEQ ID NO: 7 and SEQ ID NO:8. Protein sequence SEQ ID NO:8 appears to result from a C-terminal protein truncation of the protein sequence corresponding to SEQ ID NO: 7, and alternative amino acid C-terminal truncations can be envisioned, given the flexible nature of this protein region in homologues enzymes.

IspPro1 protease can also be expressed by integrating an expression cassette comprising a promoter sequence similar to one described in WO2017152169. Accordingly, it can be operably linked to an open reading frame encoding a signal sequence (either aprE (SEQ ID NO: 4), IspPro1 native (SEQ ID NO: 9), or other native B. subtilis signal sequence) to enable direct secretion of the the target molecule. protease, the propeptide sequence and the mature sequence of the IspPro1 protease and the terminator from the B. amyloliquefaciens BPN' gene (SEQ ID NO: 10).

IspPro1 protease can further be expressed by integrating an expression cassette for the protease into the B. licheniformis ser A locus. The introduced cassette comprises the promoter sequence described in WO2017152169 operably linked to an open reading frame encoding a signal sequence (either aprL (SEQ ID NO: 11), IspPro1 native (SEQ ID NO: 9), or other native B. licheniformis signal sequences) to target secretion of the protease, the propeptide sequence, the mature sequence of the IspPro1 protease, and the amyl, terminator sequence (SEQ ID NO: 12) from B. licheniformis. A second expression cassette for the IspPro1 protease is integrated into the B. licheniformis lysA locus. The introduced cassette comprises the promoter sequence similar to one described in WO2020112609 operably linked to a signal sequence (either aprL (SEQ ID NO: 11), IspPro1 native (SEQ ID NO: 9), or other native B. licheniformis signal sequences) to target secretion of the protease, the propeptide sequence, the mature sequence of the IspPro1, and the amyl, terminator sequence from B. licheniformis such that said B. licheniformis strain now comprises two IspPro1 expression cassettes.

The introduced IspPro1 expression cassettes for B. subtilis or B. licheniformis described in the preceding paragraphs may or may not further comprise a nucleotide sequence encoding the 3 amino acids AGK between the 3′end of the encoded signal peptide and the 5′ end of the encoded propeptide (for example SEQ ID NO: 5). The nucleotide sequence of the mature IspPro1 in the introduced cassettes may encode the protein in SEQ ID NO: 2 (precursor IspPro1), SEQ ID NO: 6 or be modified to encode truncations there of (for example SEQ ID NO: 7, SEQ ID NO: 8, or SEQ ID NO: 13).

### Example 3

### Proteolytic Activity of IspPro1 Protease

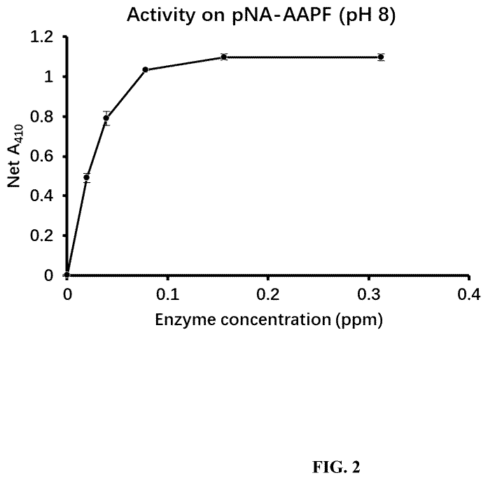

The proteolytic activity of purified IspPro1 (SEQ ID NO: 7 and SEQ ID NO: 8) was measured in 50 mM HEPES buffer (pH 8), using Suc-Ala-Ala-Pro-Phe-pNA (pNA-AAPF, Cat #L-1400.0250, BACHEM) as a substrate. Prior to the reaction, the enzyme was diluted with water to specific concentrations. The pNA-AAPF was dissolved in Dimethyl sulfoxide (DMSO, Cat #STBD2470V, SIGMA) to a final concentration of 10 mM. To initiate the reaction, 5 μL of pNA-AAPF was mixed with 85 μL of HEPES buffer in a non-binding 96-well microtiter plate (96-MTP) (Corning Life Sciences, #3641) and incubated at 40° C. for 5 min at 600 rpm in a Thermomixer (Eppendorf). Afterwards, 10 μL of the diluted enzyme (or water alone as the blank control) was added. Following a 10 min incubation in a Thermomixer at 40° C. and 600 rpm, the reaction plate was directly read at 410 nm using a SpectraMax 190. Net A₄₁₀ was calculated by subtracting the A₄₁₀ of the blank control from that of enzyme, and then plotted against different protein concentrations (from 0.02 ppm to 0.3125 ppm). Each value was the mean of triplicate assays. The proteolytic activity is shown as Net A₄₁₀. The proteolytic assay with pNA-AAPF as the substrate (FIG. 2) indicates that IspPro1 is an active protease.

### EXAMPLE 4

### pH Profile of IspPro1 Protease

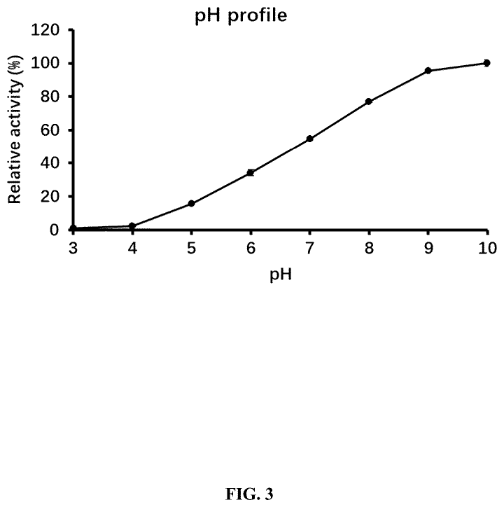

With pNA-AAPF as the substrate, the pH profile of purified IspPro1 (SEQ ID NO: 7 and SEQ ID NO: 8) was studied in 25 mM glycine/sodium acetate/HEPES buffer with different pH values (ranging from pH 3 to 10). Prior to the assay, 85 μL of 25 mM glycine/sodium acetate/HEPES buffer with a specific pH value was first mixed with 5 μL of 10 mM pNA-AAPF in a 96-MTP, and then 10 μL of water diluted enzyme (0.2 ppm for IspPro1; or water alone as the blank control) was added. The reaction was performed and analyzed as described in Example 3. Enzyme activity at each pH was reported as relative activity where the activity at the optimal pH was set to be 100%. The pH values tested were 3, 4, 5, 6, 7, 8, 9 and 10. Each value was the mean of triplicate assays. As shown in FIG. 3, IspPro1 protease is most active at alkaline conditions.

### Example 5

### Temperature Profile of IspPro1 Protease

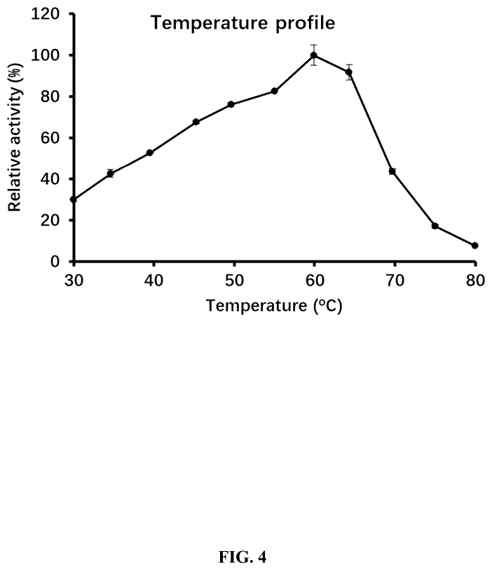

The temperature profile of purified IspPro1 (SEQ ID NO: 7 and SEQ ID NO: 8) was analyzed in 50 mM HEPES buffer (pH 8) using the pNA-AAPF assay. Prior to the reaction, 85 μL of 50 mM pH 8.0 HEPES buffer and 5 μL of 10 mM pNA-AAPF were added in a 200 μL PCR tube, which was subsequently incubated in a Peltier Thermal Cycler (BioRad) at desired temperatures (i.e. 30˜80° C.) for 5 min. After the incubation, 10 μL of water diluted enzyme (0.2 ppm for IspPro1; or water alone as the blank control) was added to the substrate to initiate the reaction. Following 10 min incubation in the Peltier Thermal Cycler at different temperatures, 80 μL of the reaction mixture was transferred to a new 96-MTP and the absorbance was read at 410 nm. The activity was reported as relative activity where the activity at the optimal temperature was set to be 100%. The tested temperatures were 30, 35, 40, 45, 50, 55, 60, 65, 70, 75 and 80° C. Each value was the mean of triplicate assays. The data in FIG. 4 suggest that IspPro1 showed an optimal activity at 60° C.

### Example 6

### Cleaning Performance of IspPro1 Protease in Automatic Dishwashing (ADW) Conditions

The cleaning performance of purified IspPro1 (SEQ ID NO: 7 and SEQ ID NO: 8) was tested using PA-S-38 microswatches (egg yolk, with pigment, aged by heating, purchased from Center for Testmaterials BV, Vlaardingen, Netherlands) at pH 10.9 using GSM-D automatic dishwashing (ADW) detergent. To prepare rinsed PA-S-38 swatches, 180 μL 10 mM CAPS buffer (pH 11) was added to 96-MTPs containing PAS38 swatches. The plates were sealed and incubated in an iEMS incubator for 30 min at 60° C., 1100 rpm. After incubation the buffer was removed, and the swatches were rinsed with water to remove any residual CAPS buffer. The plates were air dried for usage in the performance assay.

Prior to the reaction, purified IspPro1 was diluted to 200 ppm with a dilution solution containing 0.1 M NaCl, and 0.1 M CaCl₂. The reactions were performed in 3 g/L GSM-D detergent (Table 1) prepared in 21.85 GPG water hardness.

TABLE 1
Composition of GSM-D detergent

| Component | Wt % |
|---|---|
| Disilicates | 10 |
| Citrate | 30 |
| Sodium carbonate | 41 |
| Bleaching agent on | 7 |
| oxygen basis | |
| Non-ionic surfactants | 2 |
| Polycarboxylate | 6 |
| TAED | 2 |
| Sulphate, enzymes, | 2 |
| and water | |

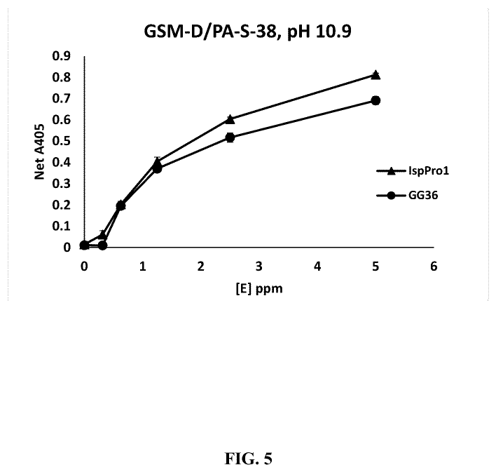

To initiate the reaction, 195 μL of the GSM-D detergent was added to the 96-MTPs placed with rinsed PA-S-38 microswatches, followed by the addition of 5 μL of diluted enzymes (or the dilution solution alone as the blank control). The 96-MTP was sealed and incubated in an incubator/shaker for 30 min at 40° C. and 1150 rpm. After incubation, 100 μL of wash liquor from each well was transferred to a new 96-MTP, and its absorbance was measured at 405 nm using a spectrophotometer. The protease activity on the model stain is shown as Net A₄₀₅, which was calculated by subtracting the A₄₀₅ of the blank control from that of enzyme. The cleaning performance of IspPro1 on PA-S-38 microswatches in GSM-D detergent at pH 10.9 was compared to a sample of Bacillus lentus WT subtilisin (GG36, SEQ ID NO: 14) and is shown in FIG. 5, where it shows superior performance.

### Example 7

### Cleaning Performance of IspPro1 Protease in Liquid Laundry Conditions

The cleaning performance of purified IspPro1 (SEQ ID NO: 7 and SEQ ID NO: 8) in Tide® liquid laundry detergent was tested using EMPA-116 microswatches (cotton soiled with blood/milk/ink; purchased from Center for Testmaterials BV, Vlaardingen, Netherlands) at pH 8.0 or pH 10.0. Prior to the reaction, the commercial liquid detergent (Tide®, Original Procter & Gamble, USA) was incubated at 95° C. for 1 hour to inactivate the enzymes present in the detergent. The heat-treated detergent was further diluted with 5 mM HEPES (pH 8.0) to a final concentration of 0.788 g/L. Meanwhile, the water hardness of the buffered liquid detergent was adjusted to 5.84 GPG. Proteolytic assays were subsequently performed to confirm the inactivation of proteases in the commercial detergents.

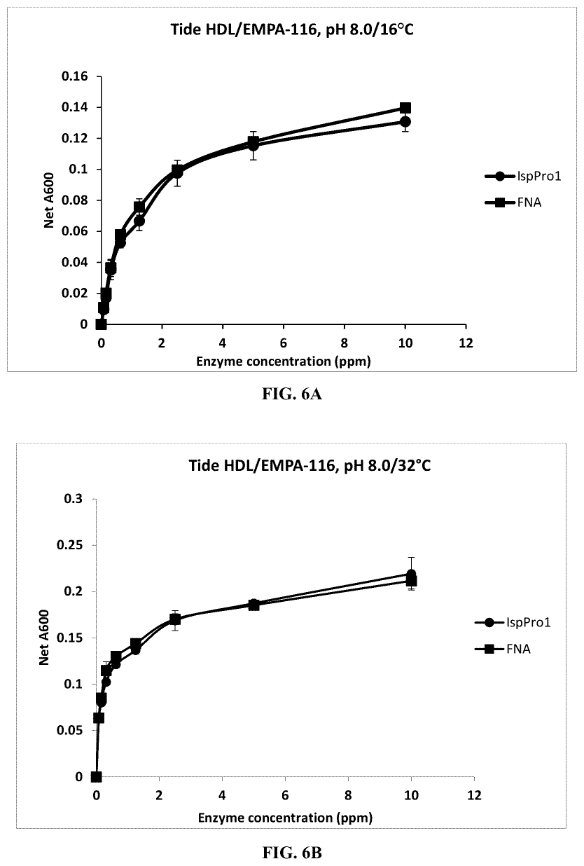

Prior to the reaction, the EMPA-116 microswatches were rinsed with water and air dried. To initiate the reaction, 190 μL of buffered detergent was added to 96-MTPs containing the rinsed EMPA-116 microswatches, followed by the addition of 10 μL of diluted enzyme (or water as blank control). The 96-MTPs were sealed and incubated for 20 min in iEMS at 32° C. and in Thermomixer at 16° C., respectively. After incubation, 100 μL of wash liquid from each well was transferred to new 96-MTPs, and then absorbance was measured at 600 nm using a spectrophotometer, which indicates the protease activity on the model stain; and Net A₆₀₀ was subsequently calculated by subtracting the A₆₀₀ of the blank control from that of the enzyme. The cleaning performance of IspPro1 on EMPA-116 microswatches at 16° C. and 32° C. was compared to FNA (BPN′-Y217L), a variant of BPN′ protease (SEQ ID NO: 15) and results are shown in FIG. 6A and FIG. 6B.

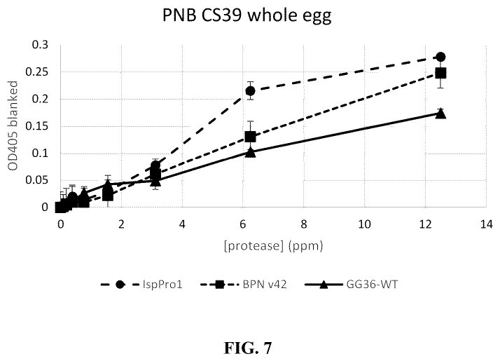

The cleaning performance of IspPro1 was also evaluated in Persil Small & Mighty Non-Bio Liquid Detergent “Persil Non-Bio” (PNB, Unilever) heavy duty liquid laundry (HDL) detergent using C-S-39 microswatches (cotton soiled with whole egg; purchased from Center for Testmaterials BV, Vlaardingen, Netherlands). PNB detergent was diluted in 5 mM HEPES, pH8.2 to a final concentration of 2.7 g/l with 12 GPG water hardness. To initiate the reactions, 195 μL of the resulting buffered liquid detergent was added to a 96-MTP well containing the C-S-39, microswatches, followed by the addition of 5 μL of water diluted enzyme (or water alone as the blank control). The 96-MTP was sealed and incubated for 25 min in iEMS at 25° C., shaking at 1150 rpm. After incubation, 100 μL of wash liquid from each well was transferred to a new 96-MTP, and the absorbance was measured at, 405 nm, using a spectrophotometer, respectively. The net absorbance was then calculated by subtracting the absorbance of the blank control from that of the corresponding enzyme treated samples. The cleaning performance of IspPro1 in PNB detergent was compared to a BPN′ variant (BPN′V42 (BPN′-S24G/S33T/S53G/N76D/S78N/S101N/G128A/Y217Q) and GG36-WT and results are shown in FIG. 7. IspPro1 showed superior performance under these test conditions.

### Example 8

### Cleaning Performance of IspPro1 protease in Powder Laundry Conditions

The cleaning performance of purified IspPro1 protein (SEQ ID NO: 7 and SEQ ID NO: 8) was evaluated in TideR powder detergent (Procter & Gamble, China; purchased from local market) using EMPA-116 (cotton soiled with blood/milk/ink) and C-S-01 microswatches (cotton soiled with blood, aged, purchased from Center for Testmaterials BV, Vlaardingen, Netherlands). For powder detergent preparation, the TideR detergent was dissolved to 2 g/L in water with 5.84 GPG water hardness and heated in a microwave to initial boiling to inactivate enzymes. Proteolytic assays were subsequently performed to confirm the inactivation of proteases in the commercial detergents.

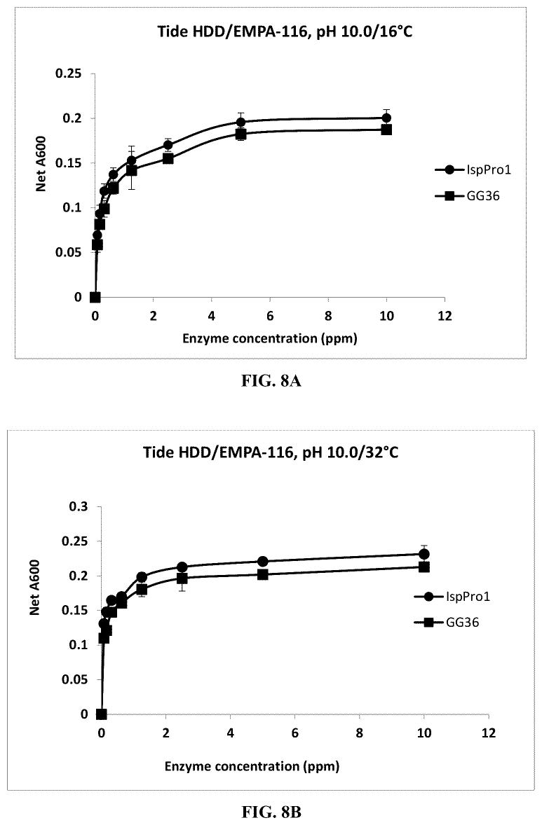

Prior to the reaction, the EMPA-116 microswatches were rinsed with water and air dried. To initiate the reaction, 190 μL of detergent was added to 96-MTPs containing the rinsed EMPA-116 microswatches, followed by the addition of 10 μL of diluted enzyme (or water as blank control). The 96-MTPs were sealed and incubated for 20 min in iEMS at 32° C. and in Thermomixer at 16° C., respectively. After incubation, 100 μL of wash liquor from each well was transferred to new 96-MTPsAbsorbance was then measured at 600 nm using a spectrophotometer, which indicates the protease activity on the model stain. TheNet A₆₀₀ was subsequently calculated by subtracting the A₆₀₀ of the blank control from that of the enzyme. The cleaning performance of IspPro1 on EMPA-116 microswatches at 16° C. and 32° C. was compared to GG36-WT and is shown in FIGS. 8A and 8B respectively, where it showed improved performance under test conditions.

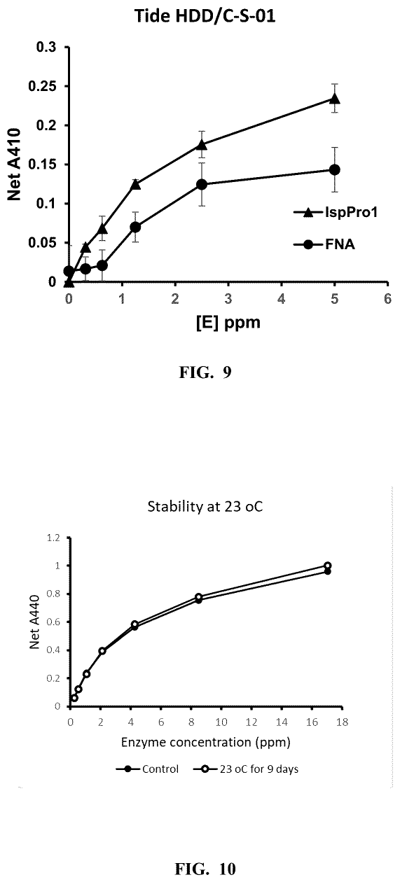

For C-S-01 microswatches, 195 μL of powder detergent solution was added to a 96-MTP well containing the C-S-01 microswatches, followed by the addition of 5 μL of water diluted enzyme (or water alone as the blank control). The 96-MTPs were sealed and incubated at 20° C. for 25 min in iEMS, shaking at 1150 rpm. After incubation, 100 μL of wash liquid from each well was transferred to a new 96-MTP, and the absorbance for C-S-01 was measured at 410 nm using a spectrophotometer. The net absorbance was then calculated by subtracting the absorbance of the blank control from that of the corresponding enzyme treated samples. The cleaning performance of IspPro1 was compared to FNA and the dose response curves are shown in FIG. 9 showing improved performance under these conditions.

### Example 9

### Stability of IspPro1 Protease

### Stability of IspPro1 Protease in Bicine-EDTA (pH 9) Buffer

The stability of purified IspPro1 (SEQ ID NO: 7 and SEQ ID NO: 8) in Bicine-EDTA buffer (pH 9.0) was determined using dimethyl casein (DMC) as the substrate. The Bicine-EDTA buffer (pH 9) contains 100 mM Bicine and 5 mM EDTA. The DMC substrate solution contains 25 mg/mL DMC in 100 mM Na₂CO₃ buffer (pH 9.2), in the presence of 100 mM NaCl. The 2,4,6-trinitrobenzene sulfonic acid (TNBSA) solution contains 0.075% TNBSA (w/v) in 100 mM Na₂CO₃ buffer (pH 9.2), in the presence of 100 mM NaCl.

To initiate the stability test in buffer, 5 μL of 0.5 mg/mL enzyme stock was added to 45 uL of Bicine-EDTA buffer in a 200 μL PCR tube. After briefly centrifuging to remove bubbles, 5 μL of the mixture were transferred to a new 200 μL PCR tube and diluted 10-fold with water to serve as an unstressed sample. The rest of the 45 μL enzyme solution was subsequently incubated at 43° C. for 5 min. After incubation, the sample was cooled to 12° C. for 30 s Next 5 μL of the resulting solution was transferred to a new 200 μL PCR tube and diluted 10-fold with water to serve as the stressed sample.

The proteolytic activity of the unstressed and stressed samples were measured using dimethyl casein (DMC) as the substrate. After mixing 10 μL of the unstressed or the stressed sample with 54 uL of the DMC substrate solution and 54 uL of the TNBSA solution in a 96-MTP, assays were carried out in a SpectraMax 190 at 40° C. The OD₄₀₅ was recorded for ten min at thirty second intervals. Absorbances between 5 and 10 minutes were used to determine enzyme activities in units of mOD₄₀₅/min. The stability of IspPro1 was compared to that of FNA under the same treatment conditions. The residual activity is shown in Table 2 as the percent of relative activity, where the activity of the unstressed sample was set to be 100% and is an average of n=4 determinations. IspPro1 showed greater stability than FNA protease under these conditions.

TABLE 2
Percent Residual Activity of IspPro1 in Bicine-EDTA buffer pH 9

| | Average residual | Std. |
|---|---|---|
| Enzyme | activity (%) | Dev |
| FNA (BPN′-Y217L) | 63.1 | 1.82 |
| IspPro1 | 88.9 | 5.00 |

### Stability of IspPro1 Protease in Tris (pH 8.0) Buffer

The extended stability of purified IspPro1 protein (SEQ ID NO: 7 and SEQ ID NO: 8) was determined by incubating samples at 23° C. for nine days and comparing its proteolytic activity to that of a sample kept frozen for the same time period. The protease activity was measured in 50 mM HEPES buffer (pH 8), using azo-casein (Cat #74H7165, Megazyme) as a substrate. Prior to the reaction, the enzyme was diluted with water to specific concentrations. Azo-casein was dissolved in 50 mM HEPES buffer (pH 8) to a final concentration of 0.75% (w/v). To initiate the reaction, 5 μl of the diluted enzyme (or water alone as the blank control) was added to the non-binding 96-MTP (Corning Life Sciences, #3641) placed on ice, followed by the addition of 95 μl of 0.75% azo-casein. After sealing the 96-MTP, the reaction was carried out in a Thermomixer (Eppendorf) at 45° C. and 650 rpm for 20 minutes. The reaction was terminated by adding 100 μl of 10% Trichloroacetic Acid (TCA). Following equilibration (5 min at the room temperature) and subsequent centrifugation (3700 g for 10 min at 4° C.), 100 μl of supernatant was transferred to a new 96-MTP, and absorbance of the supernatant was measured at 440 nm (A₄₄₀) using a SpectraMax 190. The net A₄₄₀ was calculated by subtracting the A₄₄₀ of the blank control from that of enzyme. The activity measurements are an average of duplicate assays. As seen in FIG. 10, IspPro1 is stable after 9-days incubation in 20 mM Tris (pH 8.0) supplemented with with 10% propylene glycol and 30% glycerol at room temperature.

### Storage Stability of IspPro1 Protease in Detergent

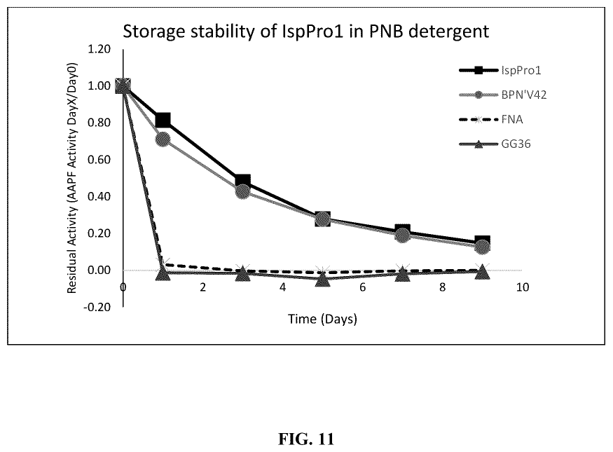

The detergent storage stability of purified IspPro1 protein (SEQ ID NO: 7 and SEQ ID NO: 8) was assessed in PNB detergent and compared to that of BPN'V42, FNA, and GG36 protease samples. Enzyme samples were mixed in 100% PNB detergent for at least 30 minutes prior to incubation at 35° C. Enzyme samples incubated in detergent were assayed for proteolytic activity at 1, 3, 5, 7, and 9 days following incubation. To measure protease activity, the detergent/enzyme mixtures were removed from incubator and mixed for 30 minutes. The samples were diluted in assay buffer using Nunc 96-well polypropylene plates. The protease activity was measured using AAPF (S7388, Sigma Aldrich) as a substrate. The pNA-AAPF was dissolved in DMSO to a stock solution of 50 mg/mL and diluted to a final concentration of 1 mg/L in 0.1M Tris buffer pH 8.6, 0.005% TWEEN-80. Five microliters of diluted detergent/enzyme mixtures were added to 95 μL of diluted AAPF substrate in a reaction plate and assayed for activity at 405 nm over 3 min using a SpectraMax plate reader in kinetic mode at RT. The protease activity was expressed as mOD/min. Residual activities were calculated as a ratio of activity day X/activity day 0 and are plotted as a function of incubation days in FIG. 11. Under these conditions, IspPro1 showed similar or somewhat higher stability when compared to a commercial liquid laundry protease, BPN' variant v42, and much higher stability than FNA and GG36 proteases.

### Example 10

### Hydrolysis and Solubilization of Corn Soy Protein with Purified IspPro1 Protease

The purified IspPro1 protease (SEQ ID NO: 7 and SEQ ID NO: 8) and the commercial product RONOZYMER ProAct protease (DSM) were tested for their ability to hydrolyze and solubilize corn soy protein using an in vitro assay mimicking the digestive tract of poultry, as described in Yu S, Cowieson A, Gilbert C, Plumstead P, Dalsgaard S.,. J. Anim. Sci. 2012, 90:1824-1832. The reaction was carried out in 96 well MTP, and consisted of 140 μL 10% (w/w) corn soy feed slurry with pH adjusted to pH 3.0, 20 μL of the protease (purified IspPro1 or ProAct) dissolved in 50 mM sodium acetate pH3.0 giving a final concentration of 500 or 1000 ppm, and 10 μL pepsin (Sigma P7000 dissolved in water at 1.69 mg/mL). The plate was incubated at 40° C. for 45 min in an iEMS Incubator/Shaker (Thermo Scientific) at 1150 rpm. At the end of the incubation 34 μL porcine pancreatin (Sigma P7545, 0.4636 mg/mL in IM sodium bicarbonate) was added and the plate was further incubated at 40° C. for 60 min in the iEMS shaking at 1150 rpm. After the incubation, the plate was centrifuged at 5° C., at 4000 rpm for 15 min. A quantity of ten microliters of supernatant was transferred to a Corning #3641 plate containing 90 μL of water (10-fold dilution). The 10-fold diluted supernatant was used to determine the extent of protein hydrolysis by the OPA method and protein solubilization was ascertained by BCA method by measuring absorbance at 340 nm and 562 nm, respectively. Protein hydrolysis using o-phthaldialdehyde (OPA) reagent was performedas described before with minor modifications (P.M. NIELSEN, D. PETERSEN, and C. DAMBMANN, J. Food Sci. 66:642-646, 2001). The OPA reagent was prepared freshly by mixing 30 mL tri-sodium phosphate (Na₃PO₄·12H₂O, 2% w/v in water with pH adjusted pH11), 0.8 mL OPA (0.4 g o-phthaldialdehyde 97% (OPA) in 10 mL 96% ethanol, saved at −20° C.), 1 mL DTT solution (0.352 g DL-dithiothreitol (DTT, 99%) in 10 mL water), and water to a final volume of 40 mL. The reagent was kept in the dark and used immediately after the preparation. 20 μL of the 10× diluted supernatant μL was mixed with 175 μL of the OPA reagent for 5 seconds and read at 340 nm after 2 minutes. Protein solubilization was measured by using the Pierce BCA Protein Assay Kit (Cat no. 23225 from Thermo Fisher Scientific). A volume of 20 μL of supernatant was mixed with 200 μL of the BCA reagent (prepared before use by mixing 50 mL BCA reagent A with 1 mL BCA regent B according to the manufacturer's instruction). The mixtures were incubated at 37° C. for 30 minutes before absorbance at 562 nm was measured. The results for corn soy protein hydrolysis and solubilization by IspPro1 and RONOZYMER ProAct protease (DSM) are shown on Table 3.

TABLE 3
Hydrolysis and protein solubilization of corn
soy feed with IspPro1 and ProAct proteases

| | Protease | OPA | OPA | BCA | BCA |
|---|---|---|---|---|---|
| | concentration | assay | assay | assay | assay |
| Sample ID | (ppm) | OD340 | Std dev | OD562 | Std dev |
| Control | NA | 0.5259 | 0.01006 | 0.7838 | 0.02743 |
| (buffer) | | | | | |
| ProAct | 500 | 0.7355 | 0.00995 | 0.9119 | 0.02209 |
| ProAct | 1000 | 0.7815 | 0.02613 | 0.9296 | 0.02334 |
| IspPro1 | 500 | 0.7340 | 0.01437 | 0.8509 | 0.01776 |
| IspPro1 | 1000 | 0.8031 | 0.03822 | 0.8821 | 0.03513 |

### Example 11

### Pepsin Stability of Purified IspPro1 Protease

The pepsin stability of purified IspPro1 protease (SEQ ID NO: 7 and SEQ ID NO: 8) was evaluated by incubating it with pepsin (Sigma, Cat. No. P7000) in 50 mM sodium acetate buffer (pH 3.0); and AAPF-pNA was used as the substrate to measure the remaining activity of IspPro1. IspPro1 and pepsin were first mixed with ratios (w/w) of 1:0, 1:25, 1:250 or 1:2500. IspPro1 was dosed at 20 ppm and the resulting mixture was subsequently incubated at 37° C. for 30 min. Meanwhile, 20 ppm IspPro1 sample was kept on ice as control. For remaining activity measurement, 5 μL of 10 mM AAPF-pNA was mixed with 85 μL of HEPES buffer (50 mM, pH 8.0) in a 96-MTP Next, 10 μL of the Milli-Q water diluted mixture (0.4 ppm for IspPro1; or H₂O alone as the blank control) was added. The reaction was performed and analyzed as described earlier in Example 3. The residual activity for a sample maintained on ice was set to be 100%. All IspPro1 samples co-incubated with porcine pepsin retained 90% or greater residual activity (data not shown).

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

What is claimed is:

1. An isolated polypeptide or active fragment thereof having serine protease activity, wherein the polypeptide comprises an amino acid sequence having at least 75%, 80%, 85%, 90%, 91%, 92%, 93%, 94%, 95%, 96%, 97%, 98%, 99%, or 100% sequence identity with an amino acid sequence selected from the group consisting of SEQ ID NO: 7, 8, and 13.

2. An isolated polypeptide having serine protease activity, wherein the polypeptide comprises an amino acid sequence having at least 75%, 80%, 85%, 90%, 91%, 92%, 93%, 94%, 95%, 96%, 97%, 98%, 99%, or 100% sequence identity with an amino acid sequence selected from the group consisting of SEQ ID NOs: 7, 8, and 13. 3 A recombinant construct comprising a regulatory sequence functional in a production host operably linked to a nucleotide sequence encoding at least one polypeptide having an amino acid sequence having at least 75%, 80%, 85%, 90%, 91%, 92%, 93%, 94%, 95%, 96%, 97%, 98%, 99%, or 100% sequence identity with an amino acid sequence selected from the group consisting of SEQ ID NOs: 7, 8, and 13.

4. The recombinant construct of claim 3, wherein said host is selected from the group consisting of fungi, bacteria, and algae.

5. A host cell comprising a recombinant nucleic acid construct comprising an isolated polynucleotide comprising a nucleotide sequence that encodes a polypeptide having serine protease activity, wherein the polypeptide has at least 75%, 80%, 85%, 90%, 91%, 92%, 93%, 94%, 95%, 96%, 97%, 98%, 99%, or 100% sequence identity with an amino acid sequence selected from the group consisting of SEQ ID NOs: 7, 8, and 13operably linked to a promoter sequence capable of controlling expression of the polynucleotide sequence. 6 The host cell of claim 5, wherein said host is selected from the group consisting of fungi, bacteria, and algae.

7. A method for producing at least one polypeptide comprising:
(a) transforming a production host with the recombinant construct comprising a regulatory sequence functional in a production host operably linked to a nucleotide sequence encoding at least one polypeptide having an amino acid sequence having at least 75%, 80%, 85%, 90%, 91%, 92%, 93%, 94%, 95%, 96%, 97%, 98%, 99%, or 100% sequence identity with an amino acid sequence selected from the group consisting of SEQ ID NOs: 7, 8, and 13; and
(b) culturing the production host of step (a) under conditions whereby at least one polypeptideis produced.

8. A method according to claim 7, wherein the polypeptide is optionally recovered from the production host.

9. A serine protease-containing culture supernatant obtained by the method of claim 7 or 8.

10. A cleaning or detergent composition comprising at least one polypeptide having an amino acid sequence having at least 75%, 80%, 85%, 90%, 91%, 92%, 93%, 94%, 95%, 96%, 97%, 98%, 99%, or 100% sequence identity with an amino acid sequence selected from the group consisting of SEQ ID NOs: 7, 8, and 13and at least one surfactant.

11. The cleaning or detergent composition of claim 10, wherein the surfactant is selected from the group consisting of a non-ionic, ampholytic, semi-polar, anionic, cationic, zwitterionic, and combinations and mixtures thereof.

12. The cleaning or detergent composition of claim 10 or 11, wherein the surfactant is a nonionic alcohol ethoxylate.

13. The cleaning or detergent composition of claims 10-12, further comprising a second polypeptide having protease activity; and, optionally, at least one additional polypeptide, wherein the at least one additional polypeptide is an enzyme selected from the group consisting of: acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, feruloyl esterase, galactanases, glucoamylases, hemicellulases, hexosaminidases, hyaluronidases, keratinases, laccases, lactases, ligninases, lipases, lipoxygenases, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof.

14. The cleaning or detergent composition of claims 10-13, wherein the composition comprises between about 0.1% to about 60%, about 1% to about 50%, or about 5% to about 40% surfactant by weight of the composition.

15. The cleaning or detergent composition of claims 10-14, wherein the composition further comprises one or more adjunct materials selected from the group consisting of builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents.

16. A cleaning or detergent composition comprising at least one polypeptide having an amino acid sequence having at least 75%, 80%, 85%, 90%, 91%, 92%, 93%, 94%, 95%, 96%, 97%, 98%, 99%, or 100% sequence identity with an amino acid sequence selected from the group consisting of SEQ ID NOs: 7, 8, and 13and at least one dispersant or dispersing polymer.

17. The cleaning or detergent composition of claim 16, wherein the composition further comprises a surfactant.

18. The cleaning or detergent composition of claim 17, wherein the surfactant is selected from the group consisting of a non-ionic, ampholytic, semi-polar, anionic, cationic, zwitterionic, and combinations and mixtures thereof.

19. The cleaning or detergent composition of claims 16-18, wherein the surfactant is a nonionic alcohol ethoxylate. 20 The cleaning or detergent composition of claims 16-19, further comprising a second polypeptide having protease activity; and, optionally, at least one additional polypeptide, wherein the at least one additional polypeptide is an enzyme selected from the group consisting of: acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, feruloyl esterase, galactanases, glucoamylases, hemicellulases, hexosaminidases, hyaluronidases, keratinases, laccases, lactases, ligninases, lipases, lipoxygenases, mannanases, metalloproteases, nucleases (e.g. deoxyribonucleases and ribonucleases), oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof.

21. The cleaning or detergent composition of claims 16-20, wherein the dispersant polymer is present in the composition in an amount from about 0.1 to about 20%, preferably from 0.2 to about 15%, more preferably from 0.3 to % by weight of the composition.

22. The composition of any one of claims 16-21, wherein said composition is selected from a laundry detergent, a fabric softening detergent, a dishwashing detergent, and a hard-surface cleaning detergent.

23. The composition of any one of claims 16-22, wherein said composition is a granular, powder, solid, bar, liquid, tablet, gel, paste and/or unit dose composition.

24. A method of cleaning, comprising contacting a surface or an item in need of cleaning with an effective amount of at least one polypeptide having serine protease activity, wherein the polypeptide comprises an amino acid sequence having at least 75%, 80%, 85%, 90%, 91%, 92%, 93%, 94%, 95%, 96%, 97%, 98%, 99%, or 100% sequence identity with an amino acid sequence selected from the group consisting of SEQ ID NO: 7, 8, and 13; and optionally further comprising the step of rinsing said surface or item after contacting said surface or item with said polypeptide.

25. A method of cleaning, comprising contacting a surface or an item in need of cleaning with a cleaning or detergent composition comprising at least one polypeptide having an amino acid sequence having at least 75%, 80%, 85%, 90%, 91%, 92%, 93%, 94%, 95%, 96%, 97%, 98%, 99%, or 100% sequence identity with an amino acid sequence selected from the group consisting of SEQ ID NOs: 7 and 87, 8, and 13 and at least one surfactant or dispersant polymer; and optionally further comprising the step of rinsing said surface or item after contacting said surface or item with said composition.

26. The method of any one of claims 24-25, wherein said item is dishware or fabric.
